(19) United States
(12) Patent Application Publication
(10) Pub. No.: US 2014/0371258 A1
(43) Pub. Date: Dec. 18, 2014

(54) Water-Soluble Polymer Conjugates of Topotecan

(71) Applicants: Xuyuan Gu, Foster City, CA (US); Jennifer Riggs-Sauthier, San Francisco, CA (US); Michael D. Bentley, Huntsville, AL (US); Tacey X. Viegas, Madison, AL (US)

(72) Inventors: Xuyuan Gu, Foster City, CA (US); Jennifer Riggs-Sauthier, San Francisco, CA (US); Michael D. Bentley, Huntsville, AL (US); Tacey X. Viegas, Madison, AL (US)

(73) Assignee: Nektar Therapeutics, San Francisco, CA (US)

(21) Appl. No.: 13/995,063

(22) PCT Filed: Dec. 16, 2011
(86) PCT No.: PCT/US11/65546

§ 371 Date: Jan. 27, 2014

Related U.S. Application Data
(60) Provisional application No. 61/424,198, filed on Dec. 17, 2010.

(51) Int. Cl. A61K 47/48; A61K 31/4745

(52) U.S. Cl. CPC A61K 47/48215; A61K 31/4745
USPC 514/283; 546/48

(57) ABSTRACT

Provided herein are water-soluble polymer conjugates of topotecan, along with compositions comprising the conjugates and related methods of making and using the same.

### CROSS-REFERENCE TO RELATED APPLICATION

This application claims the benefit of priority under 35 U.S.C. §119(e) to U.S. Provisional Patent Application No. 61/424,198 filed Dec. 17, 2010, the disclosure of which is incorporated by reference in its entirety.

### FIELD

This disclosure relates generally to conjugates of topotecan and a bi- or multi-arm water-soluble polymer, along with related compositions and methods. The linkage between the topotecan and the water-soluble polymer is releasable, thereby enabling release of the topotecan-based drug following administration of the conjugate to a patient. Moreover, the disclosure relates to and/or has application(s) in the fields of drug discovery, pharmacotherapy, physiology, organic chemistry and polymer chemistry, among others.

### BACKGROUND

Over the years, numerous methods have been proposed for improving the delivery of biologically active agents, particularly small molecule drugs. Challenges associated with the formulation and delivery of pharmaceutical agents can include poor aqueous solubility of the pharmaceutical agent, toxicity, low bioavailability, instability, and rapid in-vivo degradation, to name just a few. Although many approaches have been devised for improving the delivery of pharmaceutical agents, no single approach is without its drawbacks. For instance, commonly employed drug delivery approaches aimed at solving or at least ameliorating one or more of these problems include drug encapsulation (such as in a liposome, polymer matrix, or unimolecular micelle), covalent attachment to a water-soluble polymer such as polyethylene glycol, use of gene targeting agents, and the like.

PEGylation has also been used, albeit to a limited degree, to improve the bioavailability and ease of formulation of small molecule therapeutics having poor aqueous solubilities. For instance, water-soluble polymers such as PEG have been covalently attached to artilinic acid to improve its aqueous solubility. See, for example, U.S. Pat. No. 6,461,603. Similarly, PEG has been covalently attached to triazine-based compounds such as trimelamol to improve their solubility in water and enhance their chemical stability. See, for example, WO 02/043772. Covalent attachment of PEG to bisindolyl maleimides has been employed to improve poor bioavailability of such compounds due to low aqueous solubility. See, for example, WO 03/037384. Polymer conjugates of non-steroidal anti-inflammatory drugs (NSAIDs) and of opioid antagonists have also been prepared. See U.S. Patent Application Publication Nos. 2007/0025956 and 2006/0105046, respectively. Prodrugs of camptothecin having one or two molecules of camptothecin covalently attached to a linear polyethylene glycol have similarly been prepared (U.S. Pat. No. 5,880,131), while prodrugs of irinotecan and docetaxel having, among other things, four molecules of drug covalently attached to a multi-arm polymer, are described in U.S. Pat. No. 7,744,861 and International Patent Publication No. WO 10/019233, respectively.

The small molecule drug, topotecan (TPN), a chemotherapeutic agent, is a semi-synthetic analogue of the natural alkaloid, camptothecin, and functions as an inhibitor of topoisomerase I. Topoisomerase I relieves torsional strain in DNA during the replication, recombination, transcription, and repair of DNA by inducing reversible single strand breaks; topotecan, in turn, binds to the topoisomerase I-DNA complex and prevents religation of these single strand breaks. The chemical or IUPAC name for topotecan is (S)-10-[(dimethylamino)methyl]-4-ethyl-4,9-dihydroxy-1H-pyrano[3′,4′:6,7]indolizino[1,2-b]quinoline-3,14(4H,12H)-dione, and its structure is shown below:

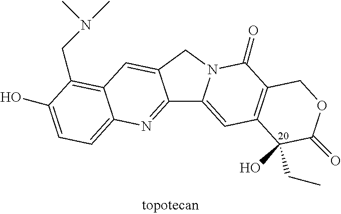 [SMILES: CC[C@@]1(O)C(=O)OCc2c1cc1n(c2=O)Cc2cc3c(CN(C)C)c(O)ccc3nc2-1]

Topotecan belongs to the camptothecin family of drugs, and possesses a basic N,N-dimethylaminomethyl functional group at carbon-9 that confers water solubility to the molecule. The chemical reactivity of the lactone ring confers biological activity to topotecan (and to the camptothecins in general). The lactone is susceptible to spontaneous reversible hydrolysis such that the intact lactone form predominates at acidic pH, while the inactive opened ring carboxylate species is favored at neutral and alkaline pH. Fassberg et al. (1992) J. Pharm. Sci. 81:676-684. Although the carboxylate form has superior water solubility in comparison to the intact, lactone form, it is 10-times less potent. Hertzberg et al. (1989) J. Med. Chem. 32:715-720. A parenteral formulation containing tartaric acid in the infusion diluent has been developed to provide a sufficiently low pH to maintain essentially all of the drug in lactone form. Shaffer et al. (1992) Anti-Cancer Drugs 3:337-345. Additionally, liposomal encapsulation of topotecan has been explored as one method for stabilizing the lactone moiety. Liu et al. (2002) Anti-Cancer Drugs 7:709-717.

Topotecan hydrochloride is marketed under the trade name, Hycamptin® (GlaxoSmithKline), and is available in both oral and injectable forms. Topotecan is currently approved for use in treating refractory ovarian cancer and small cell lung cancer sensitive disease after failure of first-line chemotherapy. Topotecan is also used, in combination with cisplatin, for stage IVB, recurrent or persistent carcinoma of the cervix that is not amenable to curative treatment with surgery or to treat cancer of the cervix that cannot be treated by surgery and/or radiation therapy.

Although shown to provide several advantages over other alkaloids for certain indications, topotecan is also associated with drawbacks as well. For example, the biological half life of topotecan in humans is much shorter than that of camptothecin sodium and other analogues (e.g., t_{1/2} for camptothecin sodium is 15.3 hours (bolus iv), while t_{1/2} for topotecan is 2.8 hours (30 min iv). Garcia-Carbonero (2002) Clin. Can. Res. 8:641. Moreover, its conventional dosing schedule is 1.5 mg/m² intravenously daily for five consecutive days every three weeks, where the standard dosing schedule has been shown clinically to be too toxic for some patients. Rowinsky (2002) The Oncologist 7(4):324-330. Additionally, administration of topotecan can cause a number of troubling side effects, including leucopenia, neutropenia, thrombocytopenia, anaemia, mucositis, and diarrhea, to name only a few. Noncumulative anaemia, neutropenia and thrombocytopenia are the dose-limiting adverse effects associated with topotecan. It would be desirous, then, if topotecan could be modified in such a way as to overcome one or more of the above-noted drawbacks of current topotecan-based therapies.

The present disclosure seeks to address these and/or other needs.

### SUMMARY

In one aspect, provided is a water-soluble polymer conjugate of topotecan having two or more topotecan molecules covalently attached, preferably releasably, to a water-soluble polymer. In one or more embodiments, a conjugate is provided having a structure encompassed by the formula:

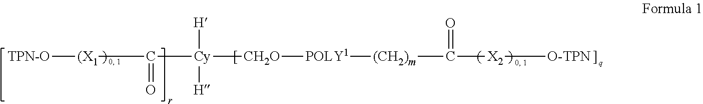

wherein:

y=0 or 1, such that when y=0, —C_{y}H′H″ is absent, and when y=1, C_{y} is present; m is a positive integer from 1 to about 12;

X₁ and X₂, when present, are each an amino acid linker, such that the amino acid carboxyl carbon of the linker is adjacent to the TPN-oxygen (O);

each POLY¹ is a water-soluble, non-peptidic polymer;

q=1, 2, 3, or 4;

r=0 or 1; and

“TPN-O-” corresponds to

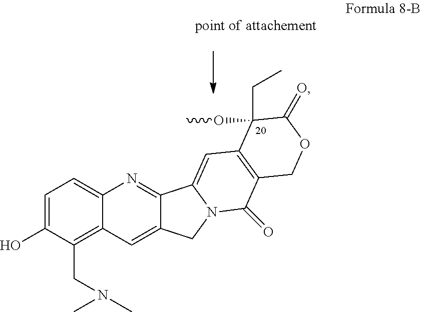

and pharmaceutically acceptable salts thereof,

where the following apply:

when r=1, q does not equal 4;

when r=0, q is selected from 2, 3, and 4, and

when r+q does not equal 4, then H′ and optionally H″ are present to bring the valence on C_{y} to four.

In one or more embodiments, X₁ and X₂ are present. (When X₁ or X₂ is present, the corresponding subscript is 1, i.e., (X₁)₁, (X₂)₁. When X₁ or X₂ is absent, the corresponding subscript is zero, i.e., (X₁)₀, (X₂)₀.

In yet one or more additional embodiments directed to X₁ and X₂, the amino acid linker comprises the structure —C(O)—CH(R″)—NH— wherein R″ is H, C1-C6 alkyl, or substituted C1-C6 alkyl. In yet another embodiment, the amino acid linker corresponds to alanine, glycine, isoleucine, leucine, phenylalanine, and valine (where it will be understood that reference to such amino acids, when considered in the context of Formula I, will conform to the remainder of the structure, e.g., absent the hydroxyl portion of its carboxylic acid functionality and absent an amino group hydrogen to suitably conform to Formula I above or any other such formula referred to herein).

In yet one or more additional embodiments, X₁ and X₂ are the same.

In one or more embodiments, provided herein is a conjugate corresponding to Formula 1 above, where r=1, q=1, and y=0, as shown in Formula 2 below, where the features of X₁, X₂, POLY₁, and m are as described above:

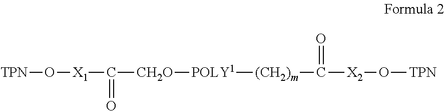

In one or more embodiments related to Formula 2, m=1.

In yet one or more further embodiments related to Formula 2, X₁ and X₂ are both present.

In yet one or more additional embodiments related to Formula 2, X₁ and X₂ are both glycine-based linkers, where X₁ corresponds to the formula: —C(O)CH₂NH— and X₂ corresponds to the formula: —NHCH₂C(O)—.

In yet one or more additional embodiments, the water soluble polymer conjugate corresponds to Formula 3:

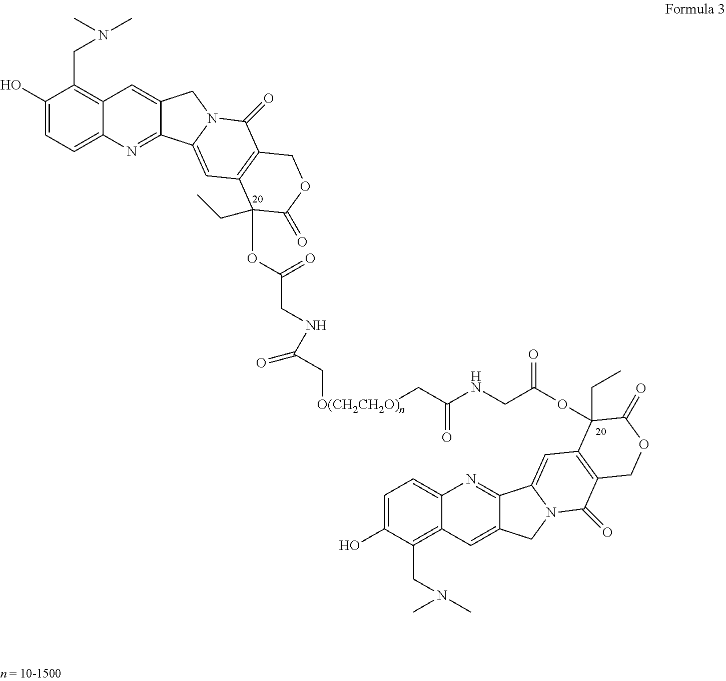

In one or more embodiments related to the foregoing, the value of n ranges from about 10 to 1500, or from about 200 to about 800.

In yet one or more additional embodiments of Formula 1, r=0, y=1, q=4, and X₂ is present as illustrated in Formula 4 below:

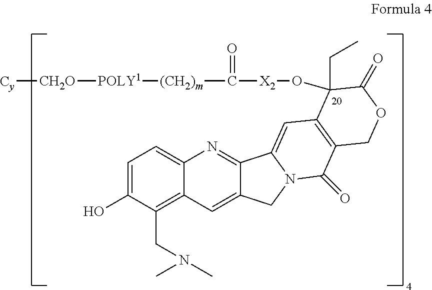

In one or more embodiments of Formula 4, m is equal to 1.

In yet one or more embodiments of Formula 4, X₂ corresponds to —NHCH₂C(O)—.

In yet one or more embodiments, the conjugate corresponds to Formula 5:

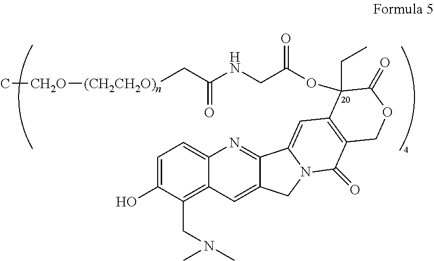

and pharmaceutically acceptable salts thereof, where n is a positive integer having a range selected from the group consisting of the following: from 10 to about 400; from about 200 to about 800.

In yet one or more embodiments, a conjugate of Formula 1 is provided, where r=0, y=1, q=2, 3 or 4, and X₂ is absent as illustrated in Formula 6 below:

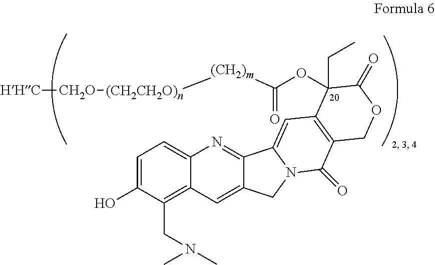

wherein n is a positive integer ranging from 10 to about 400, m is a positive integer from 3 to about 12, and, when the number of polymer arms, ˜CH₂O—(CH₂CH₂O)ₙ(CH₂)ₘC(O)—O-TPN covalently attached to central carbon CH′H″ is 2 or 3, then H′ (and optionally H″) is present to bring the valence on the central carbon to four, and pharmaceutically acceptable salts thereof.

In one or more embodiments, a conjugate-containing composition is provided, the conjugate-containing composition comprising four-arm conjugates, wherein at least 80% of the four-arm conjugates in the composition have a structure encompassed by the formula:

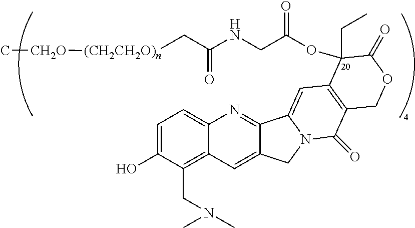

where n is a positive integer ranging from 10 to about 400, and pharmaceutically acceptable salts thereof.

In the foregoing structures, POLY¹ represents a water-soluble and non-peptidic polymer. Representative polymers include poly(alkylene glycol), poly(olefinic alcohol), poly(vinylpyrrolidone), poly(hydroxyalkylmethacrylamide), poly(hydroxyalkylmethacrylate), poly(saccharide), poly(α-hydroxy acid), poly(acrylic acid), poly(vinyl alcohol), polyphosphazene, polyoxazoline, poly(N-acryloylmorpholine), or copolymers or terpolymers thereof.

In one or more particular embodiments, POLY¹ is a polyethylene glycol. In one or more related embodiments, POLY¹ is a linear polyethylene glycol (e.g., in a bis-topotecan linear structure, or, e.g., in each arm of a multi-arm conjugate structure).

In one or more embodiments, a pharmaceutical composition is provided, the pharmaceutical composition comprising a topotecan conjugate as described herein and a pharmaceutically acceptable carrier.

In another aspect, a method is provided, the method comprising administering a topotecan conjugate as described herein (preferably in a pharmaceutical composition containing a pharmaceutically acceptable amount of the conjugate) to an individual.

Also provided herein is a method of treating cancer or any other condition responsive to treatment with topotecan by administering a polymer conjugate of topotecan as described herein.

In yet an additional aspect, in one or more embodiments thereof, a method is provided, the method comprising reacting a water-soluble, non-peptidic polymer structure having 2, 3 or 4 polymer arms (including a linear dumbbell like structure), each individual polymer arm (including in the case of a linear dumbbell configuration, each polymer terminus) having a reactive carboxylic acid group or activated ester thereof at its terminus, with “q” moles or greater of a compound having the following structure, where “q” corresponds to the number of reactive carboxylic acid or activated ester functionalities in the reactive polymeric structure:

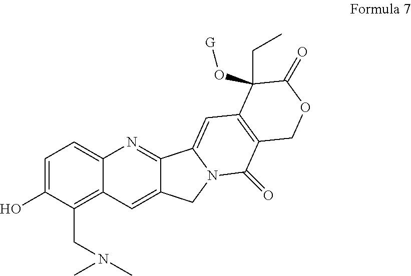

wherein G is either H or —C(O)—CHR″—NH₂, where R″ is selected from H, C1-C6 alkyl, C1-C6 alkylaryl, and substituted C1-C6 alkyl, where exemplary alkyl groups include —CH₃, —CH₂CHCH₃CH₃, —CH(CH₃)₂, —CH(CH₃)CH₂CH₃, and substituted C1-C6 alkyl groups include —(CH₂)₃—NH—C═-NH₂, —CH₂CH₂COOH, —(CH₂)₄NH₂, —(CH₂)₂SCH₃, —CH₂-Ph, -p-CH₂C₆H₄—OH, —CH₂OH, and —CHCH₃OH, where the amino group thereof is optionally in protected form.

The above methods for preparing a topotecan conjugate may include the additional steps of purifying intermediates and/or the final conjugate products, for example by size exclusion chromatography or ion exchange chromatography.

Each of the herein-described features of the invention is meant to apply equally to each and every embodiment as described herein, unless otherwise indicated.

These and other objects and features of the invention will become more fully apparent when read in conjunction with the following detailed description.

### BRIEF DESCRIPTION OF THE DRAWINGS

FIG. 1 is a graph illustrating the hydrolysis profiles of exemplary PEG-topotecan conjugates having different architectures as described in detail in Examples 1 and 2. Squares: 4-arm PEG topotecan; upright triangles: dumbbell-PEG topotecan; downward-facing triangles: mPEG-topotecan.

FIG. 2 provides a graphical presentation of the response of subcutaneously implanted HT29 human colon tumors in nude athymic mice to treatment with 4-arm PEG topotecan and dumbbell PEG topotecan when administered intravenously and compared to an untreated control group and a group treated with unmodified topotecan as described in detail in Example 4;

FIG. 3 is a graph illustrating the effects of different doses of exemplary PEG topotecan conjugates, 4-arm-PEG topotecan and dumbbell PEG topotecan, on the growth of HT29 human colon tumors implanted in athymic nude mice in comparison to a control group and a group treated with unmodified topotecan as described in Example 4;

FIG. 4 is a graph illustrating the effects of exemplary PEG topotecan conjugates, 4-arm PEG topotecan and dumbbell PEG topotecan, on the growth of NCI-H460 human lung tumors implanted in athymic nude mice in comparison to a control group and a group treated with unmodified topotecan as described in Example 5;

FIG. 5 is a graph illustrating the half-lives in plasma of exemplary PEG topotecan conjugates (linear mPEG topotecan, 4-arm PEG topotecan and dumbbell PEG topotecan) in comparison to unmodified topotecan following administration of a single IV dose in beagle dogs as described in detail in Example 6;

FIG. 6 is a graph illustrating the half-lives in plasma of topotecan released from exemplary PEG topotecan conjugates (linear mPEG topotecan, 4-arm PEG topotecan and dumbbell PEG topotecan), in comparison to unmodified topotecan following administration of a single iv dose in beagle dogs as described in detail in Example 6.

FIG. 7 is a graph illustrating the growth of H460 NSCL Carcinoma following a single IV administration of exemplary PEG topotecan conjugates (4-arm PEG topotecan and dumbbell PEG topotecan), in comparison to unmodified topotecan as described in detail in Example 7.

FIG. 8 is a graph illustrating the plasma concentration of topotecan following IV administration of exemplary PEG topotecan conjugates (4-arm PEG topotecan and dumbbell PEG topotecan), in comparison to unmodified topotecan as described in Example 7.

FIG. 9 is a graph illustrating the tumor concentration of topotecan following IV administration of exemplary PEG topotecan conjugates (4-arm PEG topotecan and dumbbell PEG topotecan), in comparison to unmodified topotecan as described in Example 7.

### DETAILED DESCRIPTION OF THE INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

### DEFINITIONS

It must be noted that, as used in this specification, the singular forms “a,” “an,” and “the” include plural referents unless the context clearly dictates otherwise. Thus, for example, reference to a “polymer” includes a single polymer as well as two or more of the same or different polymers, reference to a “conjugate” refers to a single conjugate as well as two or more of the same or different conjugates, reference to an “excipient” includes a single excipient as well as two or more of the same or different excipients, and the like.

In describing and claiming the present invention, the following terminology will be used in accordance with the definitions described below.

A “functional group” is a group that may be used, under normal conditions of organic synthesis, to form a covalent linkage between the structure to which it is attached and another structure, which typically bears a further functional group. The functional group generally includes multiple bond(s) and/or heteroatom(s). Preferred functional groups for use in the polymers of the invention are described below.

The term “reactive” refers to a functional group that reacts readily or at a practical rate under conventional conditions of organic synthesis. This is in contrast to those groups that either do not react or require strong catalysts or impractical reaction conditions in order to react (i.e., a “nonreactive” or “inert” group).

“Not readily reactive”, with reference to a functional group present on a molecule in a reaction mixture, indicates that the group remains largely intact under conditions effective to produce a desired reaction in the reaction mixture.

An “activated derivative” of a carboxylic acid refers to a carboxylic acid derivative which reacts readily with nucleophiles, generally much more readily than the underivatized carboxylic acid. Activated carboxylic acids include, for example, acid halides (such as acid chlorides), anhydrides, carbonates, and esters. Such esters include, for example, imidazolyl esters, and benzotriazole esters, and imide esters, such as N-hydroxysuccinimidyl (NHS) esters. An activated derivative may be formed in situ by reaction of a carboxylic acid with one of various reagents, e.g. benzotriazol-1-yloxy tripyrrolidinophosphonium hexafluorophosphate (PyBOP), preferably used in combination with 1-hydroxy benzotriazole (HOBT) or 1-hydroxy-7-azabenzotriazole (HOAT); O-(7-azabenzotriazol-1-yl)-N,N,N′,N′-tetramethyluronium hexafluorophosphate (HATU); or bis(2-oxo-3-oxazolidinyl)phosphinic chloride (BOP-Cl).

A “chemical equivalent” of a functional group is one that possesses essentially the same type of reactivity as the functional group. For instance, one functional group that undergoes an SN2 reaction is considered to be a functional equivalent of another such functional group.

A “protecting group” is a moiety that prevents or blocks reaction of a particular chemically reactive functional group in a molecule under certain reaction conditions. The protecting group will vary depending upon the type of chemically reactive group being protected as well as the reaction conditions to be employed and the presence of additional reactive or protecting groups in the molecule. Functional groups that may be protected include, by way of example, carboxylic acid groups, amino groups, hydroxyl groups, thiol groups, carbonyl groups and the like. Representative protecting groups for carboxylic acids include esters (such as a p-methoxybenzyl ester), amides and hydrazides; for amino groups, carbamates (such as tert-butoxycarbonyl) and amides; for hydroxyl groups, ethers and esters; for thiol groups, thioethers and thioesters; for carbonyl groups, acetals and ketals; and the like. Such protecting groups are well-known to those skilled in the art and are described, for example, in T.W. Greene and G.M. Wuts, Protecting Groups in Organic Synthesis, Third Edition, Wiley, New York, 1999, and references cited therein.

A functional group in “protected form” refers to a functional group bearing a protecting group. As used herein, the term “functional group” or any synonym thereof is meant to encompass protected forms thereof.

“PEG” or “poly(ethylene glycol)” as used herein, is meant to encompass any water-soluble poly(ethylene oxide). Typically, PEGs for use in the present invention will comprise one of the two following structures: “—(CH₂CH₂O)ₙ-” or “—(CH₂CH₂O)ₙ₋₁CH₂CH₂—,” depending upon whether or not the terminal oxygen(s) has been displaced, e.g., during a synthetic transformation. The variable (n) is 3 to 3000, and the terminal groups and architecture of the overall PEG may vary. PEGs for use in the invention include PEGs having a variety of molecular weights, structures or geometries to be described in greater detail below.

“Water-soluble,” in the context of a polymer of the invention or a “water-soluble polymer segment” is any segment or polymer that is soluble in water at room temperature. Typically, a water-soluble polymer or segment will transmit at least about 75%, more preferably at least about 95% of light, transmitted by the same solution after filtering. On a weight basis, a water-soluble polymer or segment thereof will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer or segment is about 95% (by weight) soluble in water or completely soluble in water.

An “end-capping” or “end-capped” group is an inert group present on a terminus of a polymer such as PEG. An end-capping group is one that does not readily undergo chemical transformation under typical synthetic reaction conditions. An end capping group is generally an alkoxy group, —OR, where R is an organic radical comprised of 1-20 carbons and is preferably lower alkyl (e.g., methyl, ethyl) or benzyl. “R” may be saturated or unsaturated, and includes aryl, heteroaryl, cyclo, heterocyclo, and substituted forms of any of the foregoing. For instance, an end capped PEG will typically comprise the structure “RO—(CH₂CH₂O)ₙ-”, where R is as defined above. Alternatively, the end-capping group can also advantageously comprise a detectable label. When the polymer has an end-capping group comprising a detectable label, the amount or location of the polymer and/or the moiety (e.g., active agent) to which the polymer is coupled, can be determined by using a suitable detector. Such labels include, without limitation, fluorescers, chemiluminescers, moieties used in enzyme labeling, colorimetric (e.g., dyes), metal ions, radioactive moieties, and the like.

“Non-naturally occurring” with respect to a polymer of the invention means a polymer that in its entirety is not found in nature. A non-naturally occurring polymer of the invention may however contain one or more subunits or segments of subunits that are naturally occurring, so long as the overall polymer structure is not found in nature.

“Molecular mass” in the context of a water-soluble polymer of the invention such as PEG, refers to the nominal average molecular mass of a polymer, typically determined by size exclusion chromatography, light scattering techniques, or intrinsic velocity determination in 1,2,4-trichlorobenzene. Molecular weight in the context of a water-soluble polymer, such as PEG, can be expressed as either a number-average molecular weight or a weight-average molecular weight. Unless otherwise indicated, all references to molecular weight herein refer to the weight-average molecular weight. Both molecular weight determinations, number-average and weight-average, can be measured using gel permeation chromatographic or other liquid chromatographic techniques. Other methods for measuring molecular weight values can also be used, such as the use of end-group analysis or the measurement of colligative properties (e.g., freezing-point depression, boiling-point elevation, or osmotic pressure) to determine number-average molecular weight or the use of light scattering techniques, ultracentrifugation or viscometry to determine weight-average molecular weight. The polymers of the invention are typically polydisperse (i.e., number-average molecular weight and weight-average molecular weight of the polymers are not equal), possessing low polydispersity values such as less than about 1.2, less than about 1.15, less than about 1.10, less than about 1.05, and less than about 1.03. As used herein, references will at times be made to a single water-soluble polymer having either a weight-average molecular weight or number-average molecular weight; such references will be understood to mean that the single-water soluble polymer was obtained from a composition of water-soluble polymers having the stated molecular weight.

The term “linker” is used herein to refer to a collection of atoms used to link interconnecting moieties, such as POLY¹ and the topotecan. A linker moiety may be hydrolytically stable or may include a physiologically hydrolyzable or enzymatically degradable linkage. A linker designated herein as X, e.g., X₁ or X₂, comprises a hydrolyzable linkage, where the hydrolyzable linkage is attached directly to the topotecan, such that upon hydrolysis, topotecan is released in its parent form. Preferably, X₁ and X₂ are amino acid linkers.

A “hydrolysable” bond is a relatively weak bond that reacts with water (i.e., is hydrolyzed) under physiological conditions. The tendency of a bond to hydrolyze in water will depend not only on the general type of linkage connecting two central atoms but also on the substituents attached to these central atoms. Illustrative hydrolytically unstable linkages include carboxylate ester, phosphate ester, anhydrides, acetals, ketals, acyloxyalkyl ether, imines, orthoesters, peptides and oligonucleotides.

An “enzymatically degradable linkage” means a linkage that is subject to degradation by one or more enzymes. Such a linkage requires the action of one or more enzymes to effect degradation.

A “hydrolytically stable” linkage or bond refers to a chemical bond, typically a covalent bond, that is substantially stable in water, that is to say, does not undergo hydrolysis under physiological conditions to any appreciable extent over an extended period of time. Examples of hydrolytically stable linkages include but are not limited to the following: carbon-carbon bonds (e.g., in aliphatic chains), ethers, amides, urethanes, and the like. Generally, a hydrolytically stable linkage is one that exhibits a rate of hydrolysis of less than about 1-2% per day under physiological conditions. Hydrolysis rates of representative chemical bonds can be found in most standard chemistry textbooks.

“Multi-armed” in reference to the geometry or overall structure of a polymer refers to polymer having 3 or more polymer-containing “arms,” Thus, a multi-armed polymer may possess 3 polymer arms, 4 polymer arms, 5 polymer arms, and so forth, depending upon its configuration and core structure. One particular type of highly branched polymer is a dendritic polymer or dendrimer, that, for the purposes of the invention, is considered to possess a structure distinct from that of a multi-armed polymer.

“Branch point” refers to a bifurcation point comprising one or more atoms at which a polymer splits or branches from a linear structure into one or more additional polymer arms. A multi-arm polymer may have one branch point or multiple branch points.

A “dumbbell” polymer refers generally to a linear polymer having two termini, each terminus corresponding to either a reactive functional group or an active agent. Although linear without a branch point, in some instances herein, a dumbbell polymer may be referred to as a 2-arm polymer conjugate, in reference to the two topotecan molecules at each terminus.

A “dendrimer” is a globular, size monodisperse polymer in which all bonds emerge radially from a central focal point or core with a regular branching pattern and with repeat units that each contribute a branch point. Dendrimers exhibit certain dendritic state properties such as core encapsulation, making them unique from other types of polymers.

“Substantially” or “essentially” means nearly totally or completely, for instance, 95% or greater of some given quantity.

“Alkyl” refers to a hydrocarbon chain, typically ranging from about 1 to 20 atoms in length. Such hydrocarbon chains are preferably but not necessarily saturated and may be branched or straight chain, although typically straight chain is preferred. Exemplary alkyl groups include methyl, ethyl, propyl, butyl, pentyl, 1-methylbutyl, 1-ethylpropyl, 3-methylpentyl, and the like. As used herein, “alkyl” includes cycloalkyl when three or more carbon atoms are referenced.

“Lower alkyl” refers to an alkyl group containing from 1 to 6 carbon atoms, and may be straight chain or branched, as exemplified by methyl, ethyl, n-butyl, i-butyl, t-butyl.

“Cycloalkyl” refers to a saturated or unsaturated cyclic hydrocarbon chain, including bridged, fused, or Spiro cyclic compounds, preferably made up of 3 to about 12 carbon atoms, more preferably 3 to about 8.

“Non-interfering substituents” are those groups that, when present in a molecule, are typically non-reactive with other functional groups contained within the molecule.

The term “substituted” as in, for example, “substituted alkyl,” refers to a moiety (e.g., an alkyl group) substituted with one or more non-interfering substituents, such as, but not limited to: C₃-C₈ cycloalkyl, e.g., cyclopropyl, cyclobutyl, and the like; halo, e.g., fluoro, chloro, bromo, and iodo; cyano; alkoxy, lower phenyl; substituted phenyl; and the like. For substitutions on a phenyl ring, the substituents may be in any orientation (i.e., ortho, meta, or para).

“Alkoxy” refers to an —O—R group, wherein R is alkyl or substituted alkyl, preferably C₁-C₂₀ alkyl (e.g., methoxy, ethoxy, propyloxy, etc.), preferably C₁-C₇.

As used herein, “alkenyl” refers to a branched or unbranched hydrocarbon group of 1 to 15 atoms in length, containing at least one double bond, such as ethenyl, n-propenyl, isopropenyl, n-butenyl, isobutenyl, octenyl, decenyl, tetradecenyl, and the like.

The term “alkynyl” as used herein refers to a branched or unbranched hydrocarbon group of 2 to 15 atoms in length, containing at least one triple bond, ethynyl, n-propynyl, isopropynyl, n-butynyl, isobutynyl, octynyl, decynyl, and so forth.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl.

“Heteroaryl” is an aryl group containing from one to four heteroatoms, preferably N, O, or S, or a combination thereof. Heteroaryl rings may also be fused with one or more cyclic hydrocarbon, heterocyclic, aryl, or heteroaryl rings.

“Heterocycle” or “heterocyclic” means one or more rings of 5-12 atoms, preferably 5-7 atoms, with or without unsaturation or aromatic character and having at least one ring atom which is not a carbon. Preferred heteroatoms include sulfur, oxygen, and nitrogen.

“Substituted heteroaryl” is heteroaryl having one or more non-interfering groups as substituents.

“Substituted heterocycle” is a heterocycle having one or more side chains formed from non-interfering substituents.

“Electrophile” refers to an ion, atom, or collection of atoms that may be ionic, having an electrophilic center, i.e., a center that is electron seeking, capable of reacting with a nucleophile.

“Nucleophile” refers to an ion or atom or collection of atoms that may be ionic, having a nucleophilic center, i.e., a center that is seeking an electrophilic center, and capable of reacting with an electrophile.

“Active agent” as used herein includes any agent, drug, compound, and the like which provides some pharmacologic, often beneficial, effect that can be demonstrated in-vivo or in vitro. As used herein, these terms further include any physiologically or pharmacologically active substance that produces a localized or systemic effect in a subject.

“Pharmaceutically acceptable excipient” or “pharmaceutically acceptable carrier” refers to an excipient that can be included in the compositions of the invention and that causes no significant adverse toxicological effects to the patient.

“Pharmacologically effective amount,” “physiologically effective amount,” and “therapeutically effective amount” are used interchangeably herein to mean the amount of a PEG-active agent conjugate present in a pharmaceutical preparation that is needed to provide a desired level of active agent and/or conjugate in the bloodstream or in a target tissue. The precise amount will depend upon numerous factors, e.g., the particular active agent, the components and physical characteristics of pharmaceutical preparation, intended patient population, patient considerations, and the like, and can readily be determined by one skilled in the art, based upon the information provided herein and available in the relevant literature.

“Multi-functional” in the context of a polymer means a polymer having 3 or more functional groups, where the functional groups may be the same or different, and are typically present on the polymer termini. Multi-functional polymers will typically contain from about 3-100 functional groups, or from 3-50 functional groups, or from 3-25 functional groups, or from 3-15 functional groups, or from 3 to 10 functional groups, i.e., contains 3, 4, 5, 6, 7, 8, 9 or 10 functional groups. Typically, in reference to a polymer precursor used to prepare a polymer prodrug of the invention, the polymer possesses 3 or more polymer arms having at the terminus of each arm a functional group suitable for coupling to an active agent moiety such as topotecan via a hydrolyzable linkage.

“Difunctional” or “bifunctional” as used interchangeable herein means an entity such as a polymer having two functional groups contained therein, typically at the polymer termini. When the functional groups are the same, the entity is said to be homodifunctional or homobifunctional. When the functional groups are different, the polymer is said to be heterodifunctional or heterobifunctional. Often, but not necessarily, a dumbbell structure as provided herein, in particular when conjugated, is homodifunctional.

A basic or acidic reactant described herein includes neutral, charged, and any corresponding salt forms thereof.

“Polyolefinic alcohol” refers to a polymer comprising an olefin polymer backbone, such as polyethylene, having multiple pendant hydroxyl groups attached to the polymer backbone. An exemplary polyolefinic alcohol is polyvinyl alcohol.

As used herein, “non-peptidic” refers to a polymer backbone substantially free of peptide linkages. However, the polymer may include a minor number of peptide linkages spaced along the repeat monomer subunits, such as, for example, no more than about 1 peptide linkage per about 50 monomer units.

The terms “subject,” “individual” or “patient” are used interchangeably herein and refer to a vertebrate, preferably a mammal. Mammals include, but are not limited to, murines, rodents, simians, humans, farm animals, sport animals and pets. Such subjects are typically suffering from or prone to a condition that can be prevented or treated by administration of a polymer of the invention, typically but not necessarily in the form of a polymer-active agent conjugate as described herein.

The term “about,” particularly in reference to a given quantity, is meant to encompass deviations of plus or minus five percent.

“Treatment” or “treating” of a particular condition includes: (1) preventing such a condition, i.e. causing the condition not to develop, or to occur with less intensity or to a lesser degree in a subject that may be exposed to or predisposed to the condition but does not yet experience or display the condition, (2) inhibiting the condition, i.e., arresting the development or reversing the condition.

“Optional” or “optionally” means that the subsequently described circumstance may or may not occur, so that the description includes instances where the circumstance occurs and instances where it does not.

A “small molecule” may be defined broadly as an organic, inorganic, or organometallic compound typically having a molecular weight of less than about 1000. Small molecules encompass oligopeptides and other biomolecules having a molecular weight of less than about 1000.

A “residue” refers to a portion of a compound remaining or present following a chemical reaction (whether a synthetic chemical reaction or following compound releasing-chemical reaction). For example, a polyol that is used to form a multi-arm polymer will have a “residue” of that polyol present in the multi-arm polymer.

An “active agent moiety”, or more particularly, a topotecan moiety, in reference to a conjugate as provided herein, refers to the portion or residue of the umodified parent active agent up to the covalent linkage resulting from covalent attachment of the drug (or an activated or chemically modified form thereof) to a polymer structure. Upon hydrolysis of the hydrolyzable linkage between the active agent moiety and the conjugate polymer structurer, the active agent is typically released. For example, as referred to herein, topotecan possesses the structure:

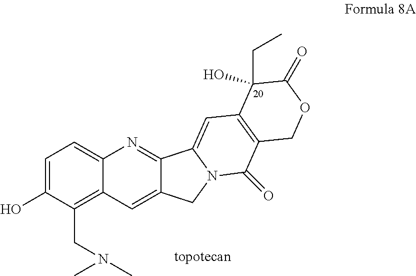 [SMILES: CC[C@@]1(O)C(=O)OCc2c1cc1n(c2=O)Cc2cc3c(CN(C)C)c(O)ccc3nc2-1]

and a topotecan moiety or topotecan residue, as provided in the instant conjugates, refers to the following structure which is absent its hydroxyl hydrogen due to covalent attachment to another moiety.

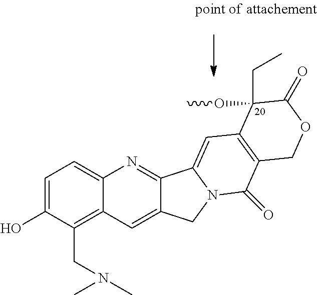

A “polyol” is an alcohol containing more than two hydroxyl groups, where the prefix “poly” in this instance refers to a plurality of a certain feature (e.g., hydroxyl functionalities) rather than to a polymeric structure. Similarly, a polythiol is a thiol containing more than two thiol (—SH) groups, and a polyamine is an amine containing more than two amino groups.

Topotecan Conjugates—Overview

As described generally above, the topotecan polymer conjugates provided herein comprise a bi- or multi-arm water-soluble and non-peptidic polymer covalently attached to at least two molecules of topotecan, preferably but not necessarily at the C-20 hydroxyl position. The conjugates described herein are typically hydrolyzable, meaning that the topotecan, attached to the polymer via a hydrolytically degradable linkage, is released over time following administration of the conjugate to a subject. Moreover, the conjugates of the invention are well-characterized, isolable, and obtained as purifiable compositions. The conjugates exhibit higher drug loading characteristics when compared to their linear polymer-based counterparts having a single drug molecule attached, thus lowering the total dosage weight needed for treatment. The polymer scaffold is effective to covalently attach two or more topotecan molecules thereto, thereby allowing a greater amount of topotecan to be administered per given weight of polymer when compared to a linear monofunctional polymer of about the same size but having only one topoptecan molecule covalently attached thereto.

The topotecan conjugate compounds provided herein exhibit efficacy in two exemplary mouse tumor models, possess an extended plasma half-life over unmodified topotecan, and were found to be more effective than topotecan in the xenograft models investigated. The foregoing results suggest that the topotecan conjugates provided herein may be of sufficient improved efficacy over topotecan per se to allow less frequent dosing than is the current practice for topotecan-based therapies. Moreover, based upon preliminary pharmacokinetics data, the topotecan conjugates provided herein may be effective in reducing the severity of the side effects typically associated with administration of unmodified topotecan. The topotecan conjugates described herein comprising about 2 or more molecules of topotecan per polymer core, when administered to a patient, may advantageously result in reduced or ameliorated side effects, which may be one or more of leucopenia, neutropenia, thrombocytopenia, anaemia, and diarrhea, when compared to the unmodified parent drug molecule. The severity of side effects of anticancer agents such as camptothecin and camptothecin-like compounds such as topotecan can be readily assessed (See, for example, Kado, et al., Cancer Chemotherapy and Pharmacology, Aug. 6, 2003). The topotecan conjugates are believed to exhibit reduced side effects as compared to the unconjugated topotecan, in part, due to the accumulation of the conjugate in the target tissue and away from other sites of likely toxicity. Each of these features of the subject topotecan conjugates will now be discussed in greater detail below.

Structural Features of the Conjugates

As described above, a topotecan conjugate as provided herein comprises a linear or a multi-arm polymer having 2, 3 or 4 molecules of topotecan attached, where the conjugate comprises the following generalized structure:

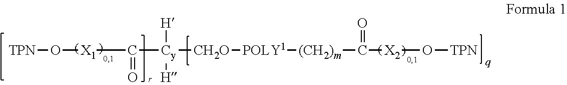

wherein: y=0 or 1 (such that when y=0, —C_{y}H′H″ is absent, and when y=1, C_{y} is present); m is a positive integer from 1 to about 12 (i.e., is selected from 1, 2, 3, 4, 5, 6, 7, 8, 9, 10, 11, and 12); X₁ and X₂, when present, are each independently an amino acid linker, such that the amino acid carboxyl carbon of the linker is adjacent to the TPN (topotecan)-oxygen (O); each POLY¹ is a water-soluble, non-peptidic polymer; q=1, 2, 3, or 4; r=0 or 1; and “TPN-O˜” corresponds to

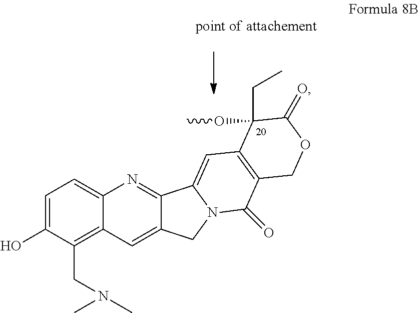

and pharmaceutically acceptable salts thereof, where the following apply: when r=1, q does not equal 4; when r=0, q is selected from 2, 3, and 4, and when r+q does not equal 4, then H′ and optionally H″ are present to bring the valence on C_{y} to four.

The polymer architecture is designed to support 2, 3 or 4 molecules of topotecan each covalently attached to the polymer scaffold via a releasable ester linkage. Each arm of the conjugate structure is independent from the other. That is to say, each of the “q” or “r” arms of the conjugate may be composed of a different Q, POLY¹, X₁, and so forth. Generally, however, each of the “q” arms of the conjugate is the same, as are X₁ and X₂ within the “q” and “r” arms.

As contemplated by the above structure, the conjugate has “q+r” number of arms, i.e., 2, 3 or 4. In one or more embodiments, the conjugates of the invention are prepared from multi-armed polymer reagents, which, in turn, are prepared from multi-arm polymers based on a multi-arm core molecule.

For example, in one approach, a polymer having 3 or 4 arms can be prepared from a corresponding multi-arm core molecule by effectively “growing” a polymer onto each terminus of a multi-arm core molecule. By way of non-limiting example, it is possible to synthesize a polymer arm onto a polyol (e.g., pentaerythritol, 2-(hydroxylmethyl)propane 1,3-diol, etc.) via an ethoxylation reaction. In another exemplary approach, a multi-arm polymer can be prepared from a multi-arm core molecule by attaching a water-soluble, non-peptidic polymer onto each terminus of a multi-arm core molecule. The principles of both approaches are described in the literature and in, for example, U.S. Pat. No. 7,026,440. The current disclosure and subject conjugates, however, is not limited with regard to the specific approach taken, so long as the conjugate is encompassed by one or more of the structures provided herein.

Organic Core, “R”

In Formula 1, the central carbon atom, C_{y}, along with its substitutents up to the polymer portion, —C_{y}(H′)(H″)[CH₂O˜]_{q}, is typically referred to as the organic core (in those embodiments in which r equals 0). In embodiments in which the “r” aim is absent, the organic radical core is typically selected from cores such as those formed from propane-1,3-diol (CH₂(CH₂OH)₂), 2-(hydroxylmethyl)propane 1,3-diol (CH(CH₂OH)₃), and 2,2-bis(hydroxymethyl)propane-1,3-diol or pentaerythritol (C(CH₂OH)₄). Preferred conjugates possess 2, 3 or 4 polymer arms.

Note that in Formula 1, —C_{y}(H′)(H″)[CH₂O˜]_{q} typically represents a residue of the core organic radical as described above. That is to say, when describing polyols, particularly by name, these molecules are being referenced in their form prior to incorporation into a water-soluble polymer-containing structure as provided herein such that the terminal alcohol function in each arm is absent its hydrogen. So, for example, when considering an illustrative conjugate of Formula 1 having a pentaerythritol “core” [C(CH₂OH)₄], the portion in Formula 1 corresponding to —C_{y}(H′)(H″)[CH₂O˜]_{q} is “C(CH₂O—) 4.” That is to say, when describing preferred organic core molecules, particularly by name, the core molecules are described in their precursor form, rather than in their radical form after removal of, for example, a proton.

In embodiments of Formula 1 in which the “r” arm is present, the organic core is as described above but additionally includes the “r” arm portion up until the oxygen of “—O-TPN,” e.g., —[(X₁)_{0,1}—C(O)]ᵣ—C_{y}(H′)(H″)[CH₂O˜]_{q}. In a linear dumbbell-type configuration, due to the lack of branching on the central carbon, C_{y}, one typically does not refer to an organic core portion of the polymer architecture.

In addition to those polyols described above and encompassed by Formula 1, additional polyols for use as the organic core include glycerol (HOCH₂CHOHCH₂OH), trimethylolpropane (C(CH₂OH)₃CH₂CH₃), reducing sugars such as sorbitol (HOCH₂CH₂OHCHOHCHOHCHOHCH₂OH), pentaerythritol, 3,3′-oxydipropane-1,2-diol (HOCH₂CHOHCH₂OCH₂CHOHCH₂OH), and glycerol oligomers such as hexaglycerol, and the like.

Water-soluble, non-peptidic-containing multi-arm polymers (used as, for example, multi-arm polymeric reagents to prepare the topotecan conjugates provided herein) are described in WO 2007/098466, WO 2010/019233 and U.S. Pat. No. 7,744,861. These references and others describe methods for preparing such multi-arm polymers. In addition, certain multi-arm polymers are available commercially from, for example, Creative PEGWorks (Winston Salem, N.C. USA), SunBio PEG-Shop (SunBio USA, Orinda, Calif.), JenKem Technology USA (Allen, Tex.), and NOF America Corporation (White Plains, N.Y.).

Linkages

The linkages, e.g., X₁ and X₂, result from the reaction of various reactive groups contained within, for example, the polymer reagent and the topotecan molecule, and, when present, serve to connect the topotecan molecule to the remainder of the polymer architecture, preferably via a releasable linkage such as an ester linkage. Illustrative linking chemistry useful for preparing the polymer conjugates of the invention can be found, for example, in Wong (1991) “Chemistry of Protein Conjugation and Crosslinking,” CRC Press, Boca Raton, Fla. and in Brinkley (1992) “A Brief Survey of Methods for Preparing Protein Conjugates with Dyes, Haptens, and Crosslinking Reagents,” in Bioconjug. Chem, 3:2013.

Typically, when present, the linkages, e.g., X₁ and X₂, each correspond to amino acids, either naturally occurring or synthetic. Again in reference to the illustrative formulas herein, X₁ and X₂ are linkers that, when covalently attached to a molecule of topotecan, comprise a hydrolyzable linkage thereto such as an ester linkage. Typically, at least one atom of the hydrolyzable linkage is contained in the topotecan molecule in its unmodified form, such that upon hydrolysis of the hydrolyzable linkage comprised within X₁ or X₂, topotecan is released. Generally speaking, the linker has an atom length of from about 3 atoms to about 25 atoms, or more preferably from about 3 atoms to about 20 atoms. Typically, the linker possesses an atom length selected from 3, 4, 5, 6, 7, 8, 9, 10, 11, 12, 13, 14, 15, 16, 17, 18, 19, and 20. For example, the linker glycine, in reference to Formula 1 or Formula 2, possesses an atom length of 3: —NH—CH₂C(O)—, where the carboxyl oxygen (C(O)O) is not counted since it is shown in the representative formulas as part of the topotecan moiety. Exemplary amino acid linkers are formed from alanine, valine, leucine, isoleucine, glycine, threonine, serine, cysteine, methionine, tyrosine, phenylalanine, tryptophan, aspartic acid, glutamic acid, lysine, arginine, histidine, proline, and non-naturally occurring amino acids. In one or more embodiments, the amino acid linker corresponds to alanine, glycine, isoleucine, leucine, phenylalanine, or valine. In one particular embodiment, the amino acid linker is glycine.

The Polymer, “POLY¹”

The topotecan conjugates provided herein can include several water-soluble, non-peptidic polymers as part of the overall structure. With respect to the conjugates, each water-soluble, non-peptidic polymer in the conjugate (e.g., POLY¹ in connection with compounds encompassed by Formula 1) is independently selected, although preferably, each water-soluble, non-peptidic polymer is of the same polymer type. That is, for example, each POLY¹ in the multi-armed conjugate is the same. Preferably, each POLY¹ in each polymer arm comprises the same polymer.

Any of a variety of water-soluble, non-peptidic polymers that are non-peptidic and water-soluble can be used in the topotecan conjugates provided herein and the disclosure is not limited in this regard. Examples of water-soluble, non-peptidic polymers include poly(alkylene glycols), copolymers of ethylene glycol and propylene glycol, poly(olefinic alcohol), poly(vinylpyrrolidone), poly(hydroxyalkylmethacrylamide), poly(hydroxyalkylmethacrylate), poly(saccharides), poly(α-hydroxy acid), poly(acrylic acid), poly(vinyl alcohol), polyphosphazene, polyoxazoline, poly(N-acryloylmorpholine), such as described in U.S. Pat. No. 5,629,384, and copolymers, terpolymers, and mixtures of any one or more of the above.

When POLY¹ is PEG, its structure typically comprises —(CH₂CH₂O)ₙ—, where n ranges from about 5 to about 400, or from about 10 to about 350, or from about 20 to about 300. Exemplary molecular weights for POLY¹ include about: 200, 250, 300, 400, 500, 600, 700, 800, 900, 1000, 1500, 2000, 3000, 4000, 5000, 6000, 7000, 7500, 8000, 9000, 10000, 12,000, 15000, 17,500, 18,000, 19,000, 20,000 daltons or greater, particularly for linear embodiments. Overall molecular weights for the topotecan polymer conjugates provided herein (that is to say, the molecular weight of the overall polymer portion as a whole) include about: 800, 1000, 1200, 1600, 2000, 2400, 2800, 3200, 3600, 4000, 5000, 6000, 8000, 10,000, 12,000, 15,000, 16,000, 20,000, 24,000, 25,000, 28,000, 30,000, 32,000, 36,000, 40,000, 45,000, 48,000, 50,000, 60,000, 80,000 daltons or greater. With respect to molecular weight ranges for the linear or multi-armed polymer, exemplary ranges include: from about 800 to about 80,000 daltons; from about 900 to about 70,000 daltons; from about 1,000 to about 40,000 daltons; from about 5,000 to about 30,000 daltons; and even from about 20,000 to about 80,000 daltons.

The Topotecan-Based Drug

The polymer conjugates provided herein include a residue of a topotecan-based compound having the following structure:

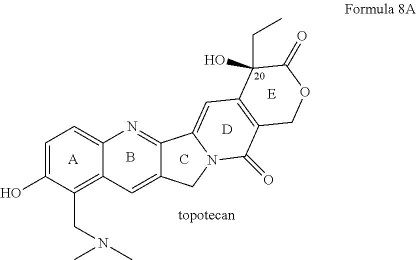

The structure shown above as Formula 8A is shown as the unmodified molecule where the lettering inside each ring indicates the designations for the various constituent rings of the overall carbocyclic ring system. Topotecan, in its residue form for use in the conjugates provided herein, will possess the following structure:

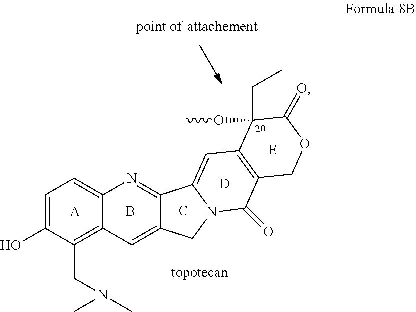

Certain exemplary forms of topotecan-based drugs (including their synthesis) are described in U.S. Pat. No. 5,004,758. Topotecan may be synthesized as described therein, or obtained from commercial sources such as Selleck Chemicals (Houston, Tex.). Topotecan is typically supplied as its mono hydrochloride salt, and may be supplied as a hydrate such as a trihydrate. Another name for topotecan is (S)-10-[(dimethylamino)methyl]-4-ethyl-4,9-dihydroxy-1H-pyrano[3′,4;6,7]indolizino[1,2-b]quinoline-3,14-(4H,12H)-dione monohydrochloride. Topotecan conjugates provided herein may also be in the form of a pharmaceutically acceptable salt.

Exemplary Conjugates

Various embodiments of topotecan conjugates are described herein as well as depicted structurally. For ease of reference, the following table is provided, although its contents are not intended to be exhaustive. Formula 1, when taken with its various variables and their individual values provided herein, is meant to explicitly describe each and every combination of variables provided as a distinct and separate embodiment.

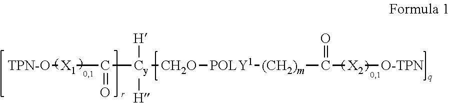

TABLE 1

| Com- | | | | | | | Com- |
|---|---|---|---|---|---|---|---|
| pound | r | q | m | y | X¹ | X₂ | ment*** |
| 1 | 1 | 1 | 1-12* | 0 | 1 (AA)** | 1 (AA) | 2 TPN, |
| | | | | | | | dumbbell |
| 2 | 1 | 2 | 1-12 | 1C_{y}H′ | 1 (AA) | 1 (AA) | 3-TPN, |
| 3 | 1 | 3 | 1-12 | 1C_{y} | 1 (AA) | 1 (AA) | 4-TPN |
| 4 | 0 | 2 | 1-12 | 1 | 0 | 1 (AA) | 2-TPN |
| | | | | C_{y}H′H″ | | | |
| 5 | 0 | 3 | 1-12 | 1 C_{y}H′ | 0 | 1 (AA) | 3-TPN |
| 6 | 0 | 4 | 1-12 | 1 C_{y} | 0 | 1 (AA) | 4-TPN |
| 7 | 0 | 2 | 1-12 | 1 | 0 | 0 | 2-TPN, |
| | | | | C_{y}H′H | | | alkanoate |
| 8 | 0 | 3 | 1-12 | 1 1 | 0 | 0 | 3-TPN, |
| | | | | C_{y}H′ | | | alkanoate |
| 9 | 0 | 4 | 1-12 | 1 C_{y} | 0 | 0 | 4-TPN, |
| | | | | | | | alkanoate |

*1-12 signifies 1, 2, 3, 4, 5, 6, 7, 8, 9, 10, 11, or 12
**AA signifies an amino acid
***in each of the above embodiments, a preferred and exemplary polymer, POLY¹, is polyethylene glycol, (CH₂CH₂O)ₙ,, where n is a positive integer in a range as described herein.

In one embodiment of Formula 1 (compound 1), variable r is equal to 1 (meaning that the “r” arm is present) and q is equal to 1. In accordance with such embodiment, the conjugate possesses the following linear structure:

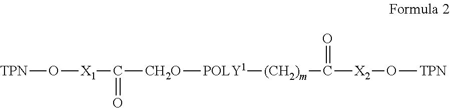

where amino acid linkers, X₁ and X₂ are present, m is selected from 1, 2, 3, 4, 5, 6, 7, 8, 9, 10, 11, and 12, and POLY¹ is a water soluble polymer such as poly(ethylene glycol), In yet another embodiment of Formula 2, m is equal to 1. Formula 2 illustrates a dumbbell polymer architecture having a topotecan molecule at each terminus. In one embodiment of compound 1, X₁ and X₂ are different amino acids. In yet another embodiment, X₁ and X₂ are the same amino acid. Representative amino acid linkers include glycine, alanine, isoleucine, leucine, phenylalanine, and valine, where, as described above, the amino acid carboxyl carbon is adjacent to the topotecan-oxygen (O-TPN). In a particular embodiment, X₁ and X₂ are both glycine. In the instance in which m is equal to 1, and X₁ and X₂ are both glycine (or any other of the same amino acid), the molecule is symmetrical as illustrated in Formula 2-i below.

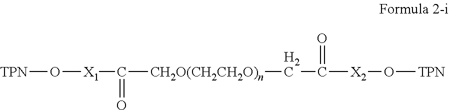

In Formula 2-i, m equals 1, such that the group to the right of the polyethylene glycol chain is a methylene group and the compound is symmetrical around the central polyethylene glycol. In one embodiment, both X₁ and X₂ are the same amino acid. Illustrative linkers include those corresponding to the structure —C(O)—CH(R″)—NH— wherein R″ is H, C1-C6 alkyl, or substituted C1-C6 alkyl. For example, X₁ and X₂ may both be alanine, glycine, isoleucine, leucine, phenylalanine, valine or the like (where it will be understood that reference to such amino acids, when considered in the context of Formula 2-i, will conform to the remainder of the structure, e.g., absent the hydroxyl portion of its carboxylic acid functionality and absent an amino group hydrogen to suitably conform to Formula 2-i above or any other such formula referred to herein). In yet another embodiment, both X₁ and X₂ are glycine such that X₁ corresponds to the formula: —C(O)CH₂NH— and X₂ corresponds to the formula: —NHCH₂C(O)—.

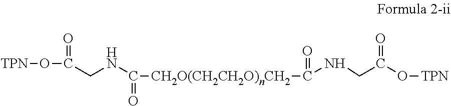

In yet another embodiment, as embodied by ‘compound 2’, r is equal to 1 and q is equal to 2, such at the conjugate possesses three topotecan molecules covalently attached thereto. See Formula 9. Two of the topotecan molecules emanate from polymer ‘arms’ extending from the central central carbon, Cy, while the third topotecan molecule is covalently attached to the central carbon via an intervening amino acid linker connected to the carbonyl carbon. The values for POLY¹, X₁, X₂, and m are as described above.

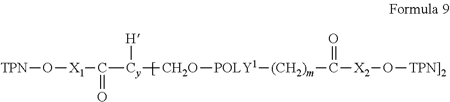

In one or more embodiments, each POLY¹ is polyethylene glycol, and X₁ and each X₂ corresponds to the same amino acid. In yet another embodiment, m is equal to 1.

In yet another embodiment as encompassed by compound 3, r is equal to 1 and q is equal to 3, such that the conjugate possesses four topotecan molecules covalently attached thereto. Three of the topotecan molecules emanate from polymer “arms” extending from the central central carbon, Cy, while the fourth topotecan molecule is covalently attached to the central carbon via an intervening amino acid linker connected to the carbonyl carbon. The values for POLY¹, X₁, X₂, and m are as described above. See Formula 10 below.

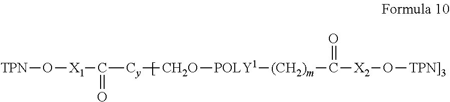

In one or more embodiments, each POLY¹ is polyethylene glycol, and X₁ and each X₂ corresponds to the same amino acid, such as glycine. In yet another embodiment, m is equal to 1.

In one or more additional embodiments as encompassed by compound 4, r is equal to 0 and q is equal to 2, such that the conjugate possesses on average two topotecan molecules covalently attached thereto as shown in Formula 11 below.

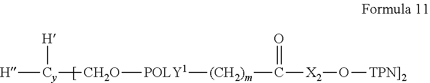

The two topotecan molecules emanate from polymer “arms” extending from the central central carbon, Cy. Representative values for POLY¹, X₂, and m are as described elsewhere herein. In one or more embodiments, each POLY¹ is polyethylene glycol, and each X₂ corresponds to the same amino acid, such as glycine. In yet another embodiment, m is equal to 1.

In one or more additional embodiments as encompassed by compound 5, r is equal to 0 and q is equal to 3, such that the conjugate possesses on average three topotecan molecules covalently attached thereto as shown in Formula 12 below.

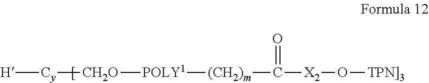

The three topotecan molecules emanate from polymer “arms” extending from the central central carbon, Cy. Representative values for POLY¹, X₂, and m are as described elsewhere herein. In one or more embodiments, each POLY¹ is polyethylene glycol, and each X₂ corresponds to the same amino acid, such as glycine. In yet another embodiment, m is equal to 1.

In one or more additional embodiments as encompassed by compound 6, r is equal to 0 and q is equal to 4, such that the conjugate possesses on average four topotecan molecules covalently attached thereto as shown in Formula 13 below. (Formula 13 is essentially identical to Formula 4, with the exception that in Formula 13, an abbreviation is used for the topotecan molecule).

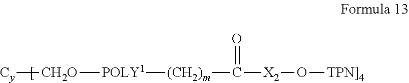

The four topotecan molecules emanate from polymer ‘arms’ extending from the central central carbon, Cy. Representative values for POLY¹, X₂, and m are as described elsewhere herein. In one or more embodiments, each POLY¹ is polyethylene glycol, and each X₂ corresponds to the same amino acid, such as glycine. In yet another embodiment, m is equal to 1.

In one or more additional embodiments as encompassed by compound 7, r is equal to 0, q is equal to 2, and X₂ is absent. The resulting conjugate possesses on average two topotecan molecules covalently attached thereto as shown in Formula 14 below.

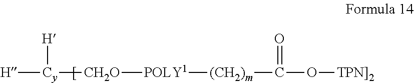

The above structure possesses a topotecan molecule at each terminus. Representative values for POLY¹ and m are as described elsewhere herein. In one or more embodiments, each polymer “arm” is the same. In one or more further embodiments, each POLY¹ is polyethylene glycol, and m is selected from 1, 2, 3, 4, 5, and 6. In yet one or more additional embodiments, m is 3.

In one or more additional embodiments as encompassed by compound 8, r is equal to 0, q is equal to 3, and X₂ is absent. The resulting conjugate possesses on average three topotecan molecules covalently attached thereto as shown in Formula 15 below.

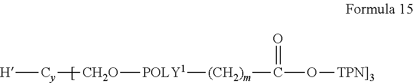

The three topotecan molecules emanate from polymer ‘arms’ extending from the central central carbon, Cy. Representative values for POLY¹ and m are as described elsewhere herein. In one or more embodiments, each of the polymer “arms” is the same. In one or more additional embodiments, each POLY¹ is polyethylene glycol, and m is selected from 1, 2, 3, 4, 5, and 6. In yet one or more additional embodiments, m is 3.

In one or more additional embodiments as encompassed by compound 9, r is equal to 0, q is equal to 4, and X₂ is absent. The resulting conjugate possesses on average four topotecan molecules covalently attached thereto as shown in Formula 16 below.

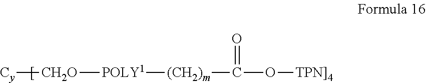

The four topotecan molecules emanate from polymer ‘arms’ extending from the central central carbon, Cy. Representative values for POLY¹ and m are as described elsewhere herein. In one or more embodiments, each of the polymer “arms” is the same. In one or more additional embodiments, each POLY¹ is polyethylene glycol, and m is selected from 1, 2, 3, 4, 5, and 6. In yet one or more additional embodiments, m is 3.

Method of Forming a Topotecan Conjugate

The conjugates provided herein can be prepared using conventional synthetic methodologies of organic chemistry, and the disclosure is not limited with respect to the manner in which the topotecan conjugates are made.

In one or more embodiments, the conjugates of the invention are prepared from multi-armed polymer reagents having 2, 3 or 4 polymer arms, which, in turn, are prepared from multi-arm polymers based on a multi-arm core molecule having 2, 3 or 4 arms. For example, in one approach, a multi-arm polymer can be prepared from a multi-arm core molecule by effectively “growing” a polymer onto each terminus of a multi-arm core molecule. By way of non-limiting example, it is possible to synthesize a polymer arm onto a polyol (e.g., pentaerythritol, diglycerol, etc.) via an ethoxylation reaction. In another exemplary approach, a multi-arm polymer can be prepared from a multi-arm core molecule by attaching a water-soluble, non-peptidic polymer onto each terminus of a multi-arm core molecule. The principles of both approaches are described in the literature and in, for example, U.S. Pat. No. 7,026,440. The invention, however, is not limited with regard to the specific approach taken, so long as the conjugate is encompassed by one or more of the structures provided herein.

In one approach for preparing a topotecan conjugate as provided herein, a linear bifunctional polymer reagent or a polymer reagent having 2, 3 or 4 polymer arms (which can be be obtained from commercially available sources, such as Creative PEGWorks, SunBio PEG-Shop, JenKem Technology USA, and NOF America Corporation, or prepared in accordance with descriptions provided in the literature) is contacted, under conjugation conditions, with an excess (typically at least a molar excess of the number of “q+r” arms of the reagent) of topotecan. Conjugation conditions are those conditions of temperature, pH, time, solvent, and so forth that allow for covalent attachment between a reactive group of the reagent to a functional group of the topotecan or modified topotecan. Exemplary conjugation conditions between a given polymer reagent bearing a reactive group and a corresponding functional group of topotecan or modified topotecan will be known to one of ordinary skill in the art in view of the disclosure provided herein. See, for example, Poly(ethylene glycol) Chemistry and Biological Applications, American Chemical Society, Washington, D.C. (1997).

A polymer reagent suitable for use in connection with conjugation conditions will typically one or more reactive groups selected from the group consisting of: N-succinimidyl carbonate (see e.g., U.S. Pat. Nos. 5,281,698, 5,468,478), amine (see, e.g., Buckmann et al. Makromol. Chem. 182:1379 (1981), Zalipsky et al. Eur. Polym. J. 19:1177 (1983)), hydrazide (See, e.g., Andresz et al. Makromol. Chem. 179:301 (1978)), succinimidyl propionate and succinimidyl butanoate (see, e.g., Olson et al. in Poly(ethylene glycol) Chemistry & Biological Applications, pp 170-181, Harris & Zalipsky Eds., ACS, Washington, D.C., 1997; see also U.S. Pat. No. 5,672,662), succinimidyl succinate (See, e.g., Abuchowski et al. Cancer Biochem. Biophys. 7:175 (1984) and Joppich et al., Makromol. Chem. 180:1381 (1979), succinimidyl ester (see, e.g., U.S. Pat. No. 4,670,417), benzotriazole carbonate (see, e.g., U.S. Pat. No. 5,650,234), glycidyl ether (see, e.g., Pitha et al. Eur. J. Biochem. 94:11 (1979), Elling et al., Biotech. Appl. Biochem. 13:354 (1991), oxycarbonylimidazole (see, e.g., Beauchamp, et al., Anal. Biochem. 131:25 (1983), Tondelli et al. J. Controlled Release 1:251 (1985)), p-nitrophenyl carbonate (see, e.g., Veronese, et al., Appl. Biochem. Biotech., 11:141 (1985); and Sartore et al., Appl. Biochem. Biotech., 27:45 (1991)), aldehyde (see, e.g., Harris et al. J. Polym. Sci. Chem. Ed. 22:341 (1984), U.S. Pat. No. 5,824,784, U.S. Pat. No. 5,252,714), maleimide (see, e.g., Goodson et al. Bio/Technology 8:343 (1990), Romani et al. in Chemistry of Peptides and Proteins 2:29 (1984)), and Kogan, Synthetic Comm. 22:2417 (1992)), orthopyridyl-disulfide (see, e.g., Woghiren, et al. Bioconj. Chem. 4:314 (1993)), acrylol (see, e.g., Sawhney et al., Macromolecules, 26:581 (1993)), vinylsulfone (see, e.g., U.S. Pat. No. 5,900,461). Exemplary conjugation conditions (including conditions of temperature, pH, time and solvent) for a given reactive group can be readily determined by one of skill in the art of polymer or organic synthesis, and are also described in the references provided above.

In the exemplary syntheses provided in Example 1, the C-20 hydroxyl group of topotecan is modified by attachment to an amino acid such as from alanine, valine, leucine, isoleucine, glycine, threonine, serine, cysteine, methionine, tyrosine, phenylalanine, tryptophan, aspartic acid, glutamic acid, lysine, arginine, histidine, proline, and non-naturally occurring amino acids in the presence of a suitable coupling agent. The amino acid will typically possess a protected amino group and an available carboxylic acid group for reaction with the topotecan hydroxyl function.

Suitable protecting groups include t-BOC and FMOC (9-flourenylmethloxycarbonyl), among others. t-BOC is stable at room temperature and easily removed with dilute solutions of acid, e.g., trifluoroacetic acid in dichloromethane. FMOC is a base labile protecting group that is easily removed by concentrated solutions of amines (usually 20-55% piperidine in N-methylpyrrolidone).

The carboxyl group of a protected amino acid such as N-protected glycine reacts with the 20-hydroxyl group of topotecan in the presence of a coupling agent (e.g., dicyclohexylcarbodiimide (DCC)) and a base catalyst (e.g., dimethylaminopyridine (DMAP) or other suitable base) to provide N-protected amino-acid modified topotecan, e.g., t-Boc-glycine-topotecan, which may optionally be in salt form. Preferably, each reaction step is conducted under an inert atmosphere.

In a subsequent step, the amino protecting group, e.g., t-BOC (N-tert-butoxycarbonyl) or other suitable protecting group is removed, e.g., by treatment with trifluoroacetic acid (TFA) or other suitable reagent under appropriate reaction conditions to provide deprotected amino-acid modified topotecan such as 20-glycine-topotecan. The amino-acid modified topotecan is then coupled to an appropriate polymer reagent, e.g., 4-arm pentaerythritolyl-PEG-succinimide or linear difunctional PEG succinimide (or any other similarly activated ester counterpart) in the presence of a coupling agent (e.g., hydroxybenzyltriazole (HOBT)) and a base (e.g., DMAP, trimethyl amine, triethyl amine, etc.), to form the desired conjugate. Reaction yields for the polymer coupling reaction are typically high, greater than about 90%. In certain instances, a quantitative conversion may be achieved.

In yet another synthetic approach, e.g., to prepare an alkanoate-linked compound such as compounds 7, 8, or 9 in Table 1, one may functionalize a suitable hydroxyl-terminated polymeric reagent by reaction with a protected haloalkanoic acid in the presence of a strong base to form the corresponding alkanoic acid functionalized polymer reagent. The resulting polymer alkanoic acid is then coupled to topotecan, e.g., the C-20 hydroxyl group therein, using a suitable condensing agent such as diisopropylcarbodiimide (DIC).

In yet another approach to prepare an alkanoate-linked compound, the C-20 hydroxyl group of topotecan is functionalized to contain an alkanoic acid moiety, followed by covalent attachment to the polymer reagent to provide a conjugate as described herein. Many variations on the methods described can be envisioned by one skilled in the art.

The topotecan conjugate is recovered, e.g., by precipitation with ether (e.g., methyl tert-butyl ether, diethyl ether) or other suitable solvent. The product may be further purified by any suitable method. Methods of purification and isolation include precipitation followed by filtration and drying, recrystallization, as well as chromatography. Suitable chromatographic methods include gel filtration chromatography, ion exchange chromatography, and Biotage Flash chromatography. One preferred method of purification is recrystallization. For example, the conjugate is dissolved in a suitable single or mixed solvent system (e.g., isopropanol/methanol), and then allowed to crystallize. Recrystallization may be conducted multiple times, and the crystals may also be washed with a suitable solvent in which they are insoluble or only slightly soluble (e.g., methyl tert-butyl ether or methyl-tert-butyl ether/methanol). The purified product may optionally be further air or vacuum dried.

Topotecan Conjugate Salts

The topotecan conjugates may be used in their basic, non-salt form. In addition, the conjugates may be used in the form corresponding to a pharmaceutically acceptable salt of the conjugate, and any reference to the conjugates of the invention herein is intended to include pharmaceutically acceptable salts. If used, a salt of a compound as described herein should be both pharmacologically and pharmaceutically acceptable, but non-pharmaceutically acceptable salts may conveniently be used to prepare the free active compound or pharmaceutically acceptable salts thereof and are not excluded from the scope of this invention. Such pharmacologically and pharmaceutically acceptable salts can be prepared by reaction of the compound with an organic or inorganic acid, using standard methods detailed in the literature. Examples of useful salts include, but are not limited to, those prepared from the following acids: hydrochloric, hydrobromic, sulfuric, nitric, phosphoric, maleic, acetic, salicyclic, p-toluenesulfonic, trifluoroacetic, tartaric, citric, methanesulfonic, formic, malonic, succinic, naphthalene-2-sulphonic and benzenesulphonic, and the like. Also, pharmaceutically acceptable salts can be prepared as alkaline metal or alkaline earth salts, such as sodium, potassium, or calcium salts of a carboxylic acid group.

Compositions of Conjugates of the Invention

In certain instances, due to incomplete conversions, less than 100% yields, and other unavoidable complications routinely encountered during chemical syntheses, exemplary compositions of topotecan conjugates are those wherein at least 80% of the conjugates in the composition have a structure as set forth previously herein, e.g., in any one of the representative formulas provided herein and pharmaceutically acceptable salts thereof.

Compositions comprising a topotecan conjugate as provided herein and designated in formulaic fashion as having an “X₁—O-Tpn or X₂—O-Tpn” at two or more termini can, in one or more embodiments, be characterized as compositions comprising those same conjugates, wherein at least 90% of the conjugates in the composition possess a structure encompassed by the following:

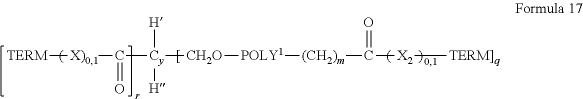

wherein:

y=0 or 1, such that when y=0, —C_{y}H′H″ is absent, and when y=1, C_{y} is present;

m is a positive integer from 1 to about 12;

X₁ and X₂, when present, are each an amino acid linker corresponding to —NH—C(R″)—C(O)—, where R″ is H, C1-C6 alkyl, or substituted C1-C6 alkyl, such that the amino acid carbonyl carbon of the linker is adjacent to the TPN-oxygen (O);

each POLY¹ is a water-soluble, non-peptidic polymer;

q=1, 2, 3, or 4;

r=0 or 1; and

“TPN-O˜” corresponds to

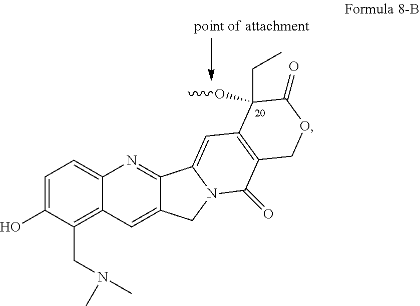

and pharmaceutically acceptable salts thereof,

where the following apply:

when r=1, q does not equal 4;

when r=0, q is selected from 2, 3, and 4,

when r+q does not equal 4, then H′ and optionally H″ are present to bring the valence on C_{y} to four, and

TERM-(X₁)₀ and —(X₂)₀-TERM, in each instance, is selected from the group

consisting of

### —OH, —OCH₃,

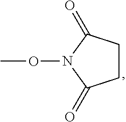

and TERM-(X₁)₁ and —(X₂)₁-TERM, in each instance, is selected from the group consisting of —NH—C(R″)—C(O)—OH, —NH—C(R″)—C(O)—OCH₃, where R″ is H, C1-C6 alkyl, or substituted C1-C6 alkyl, and —NH—C(R″)—C(O)—O-Tpn, and

(ii) for each TERM in the at least 90% of the conjugates in the composition, at least 90% thereof are —(X₁)_{0,1}—O-Tpn or (X₂)_{0,1}—O-Tpn.

Typically, although not necessarily, the number of r+q arms will correspond to the number of active agent molecules in the conjugate. That is to say, in the case of a polymer reagent having a certain number of arms, each having a reactive functional group (e.g., carboxy, activated ester such as succinimidyl ester, benzotriazolyl carbonate, and so forth) at its terminus, the optimized number of topotecan molecules that can be covalently attached thereto in the corresponding conjugate is most desirably “r+q.” That is to say, the optimized conjugate is considered to have a topotecan substitution value of 1.00(q+r) (or 100%). In a preferred embodiment, the topotecan polymer conjugate is characterized by a degree of substitution of 0.90(r+q) (or 90%) or greater. Preferred conjugate substitution amounts satisfy one or more of the following: 0.92(r+q) or greater; 0.93(r+q) or greater; 0.94(r+q) or greater; 0.95(r+q) or greater; 0.96(r+q) or greater; 0.97(r+q) or greater; 0.98(r+q) or greater; and 0.99(r+q) or greater.

By way of further explanation, a composition comprising a topotecan water soluble polymer conjugate may comprise a mixture of topotecan conjugates having one topotecan molecule therein, having two topotecan molecules therein, having three topotecan molecules therein, or four topotecan molecules therein. The resulting composition will possess an overall topotecan substitution value, averaged over the conjugate species contained in the composition.

As an illustration, in an instance in which the multi-armed topotecan polymer conjugate contains four arms, the idealized value of the number of covalently attached topotecan molecules per conjugate is four, and—with respect to describing the average in the context of a composition of such conjugates—there will be a value (i.e., percentage) of topotecan molecules loaded onto the polymer core ranging from about 90% to about 100% of the idealized value. That is to say, the average number of topotecan molecules covalently attached to the polymer core is typically 93%, 94%, 95%, 96%, 97%, 98%, 99%, up to 100% of the fully substituted value.

Pharmaceutical Compositions

Also provided are pharmaceutical formulations or compositions, both for veterinary and for human medical use, which comprise one or more topotecan polymer conjugates as provided herein with one or more pharmaceutically acceptable carriers, and optionally other therapeutic ingredients, stabilizers, or the like. The carrier(s) must be pharmaceutically acceptable in the sense of being compatible with the other ingredients of the formulation and not unduly deleterious to the recipient thereof. The compositions of the invention may also include polymeric excipients/additives or carriers, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80”, and pluronics such as F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), and in “Handbook of Pharmaceutical Excipients”, Third Ed., Ed. A. H. Kibbe, Pharmaceutical Press, 2000.

The topotecan conjugates may be formulated in compositions including those suitable for oral, rectal, topical, nasal, ophthalmic, or parenteral (including intraperitoneal, intravenous, subcutaneous, or intramuscular) administration. The compositions may conveniently be presented in unit dosage form and may be prepared by any of the methods well known in the art of pharmacy. All methods include the step of bringing the conjugate into association with a carrier that constitutes one or more accessory ingredients. In general, the compositions are prepared by bringing the topotecan conjugate into association with a liquid carrier to form a solution or a suspension, or alternatively, bringing the topotecan conjugate into association with formulation components suitable for forming a solid, optionally a particulate product, and then, if warranted, shaping the product into a desired delivery form. Solid formulations, when particulate, will typically comprise particles with sizes ranging from about 1 nanometer to about 500 microns. In general, for solid formulations intended for intravenous administration, particles will typically range from about 1 nm to about 10 microns in diameter. Particularly preferred are sterile, lyophilized compositions that are reconstituted in an aqueous vehicle prior to injection.

A preferred formulation is a solid formulation comprising a topotecan conjugate as provided herein. The solid formulation comprises sorbitol and lactic acid, and is typically diluted with 5% dextrose injection or 0.9% sodium chloride injection prior to intravenous infusion.

The amount of topotecan polymer conjugate in the formulation will vary depending upon the activity of the conjugate, its particular form (active lactone or inactive carboxylate), the molecular weight of the conjugate, and other factors such as dosage form, target patient population, and other considerations, and will generally be readily determined by one skilled in the art. The amount of conjugate in the formulation will be that amount necessary to deliver a therapeutically effective amount of camptothecin compound to a patient in need thereof to achieve at least one of the therapeutic effects associated with the topotecan, e.g., treatment of cancer. In practice, this will vary widely depending upon the particular conjugate, its activity, the severity of the condition to be treated, the patient population, the stability of the formulation, and the like. Compositions will generally contain anywhere from about 1% by weight to about 99% by weight conjugate, typically from about 2% to about 95% by weight conjugate, and more typically from about 5% to 85% by weight conjugate, and will also depend upon the relative amounts of excipients/additives contained in the composition. More specifically, the composition will typically contain at least about one of the following percentages of conjugate: 2%, 5%, 10%, 20%, 30%, 40%, 50%, 60%, or more by weight.

Compositions suitable for oral administration may be presented as discrete units such as capsules, cachets, tablets, lozenges, and the like, each containing a predetermined amount of the topotecan conjugate as a powder or granules; or a suspension in an aqueous liquor or non-aqueous liquid such as a syrup, an elixir, an emulsion, a draught, and the like.

A tablet may be made by compression or molding, optionally with one or more accessory ingredients. Compressed tablets may be prepared by compressing in a suitable machine, with the active compound being in a free-flowing form such as a powder or granules which is optionally mixed with a binder, disintegrant, lubricant, inert diluent, surface active agent or dispersing agent. Molded tablets comprised with a suitable carrier may be made by molding in a suitable machine.

A syrup may be made by adding the topotecan conjugate to a concentrated aqueous solution of a sugar, for example sucrose, to which may also be added any accessory ingredient(s). Such accessory ingredients may include flavorings, suitable preservatives, an agent to retard crystallization of the sugar, and an agent to increase the solubility of any other ingredient, such as polyhydric alcohol, for example, glycerol or sorbitol.

Formulations suitable for parenteral administration conveniently comprise a sterile aqueous preparation of the prodrug conjugate, which can be formulated to be isotonic with the blood of the recipient.

Nasal spray formulations comprise purified aqueous solutions of the topotecan conjugate with preservative agents and isotonic agents. Such formulations are preferably adjusted to a pH and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye.

Topical formulations comprise the topotecan conjugate dissolved or suspended in one or more media such as mineral oil, petroleum, polyhydroxy alcohols or other bases used for topical formulations. The addition of other accessory ingredients as noted above may be desirable.

Pharmaceutical formulations are also provided which are suitable for administration as an aerosol by inhalation. These formulations comprise a solution or suspension of the desired topotecan polymer conjugate or a salt thereof. The desired formulation may, for example, be placed in a small chamber and nebulized. Nebulization may be accomplished by compressed air or by ultrasonic energy to form a plurality of liquid droplets or solid particles comprising the conjugates or salts thereof.

Methods of Use

The topotecan conjugates provided herein can be used to treat or prevent any condition responsive to unmodified topotecan in any animal, particularly in mammals, including humans.

The topotecan conjugates are particularly useful as anticancer agents, i.e., have been shown to be effective in significantly reducing the growth of certain solid tumors as evidenced by representative lung and colon cancers in in-vivo studies, among others. Examples 4 and 5 illustrate the effectiveness of illustrative topotecan polymer conjugates in the treatment of colorectal and lung cancer, respectively, based upon in-vivo xenograft model results.

The topotecan polymer conjugate compounds provided herein may be used to treat any one or more of the following: breast cancer, ovarian cancer, colon cancer, colorectal cancer, prostate cancer, gastric cancer, malignant melanoma, small cell lung cancer, non-small cell lung cancer, thyroid cancers, kidney cancer, cancer of the bile duct, brain cancer, cancer of the head and neck, multiple myeloma, myelodysplastic sundrome, neuroblastoma, pancreatic cancer, retinoblastoma, rhabdomyosarcoma, Ewing's sarcoma, lymphomas, leukemias, rhabdomyosarcoma, neuroblastoma, and the like. The instant compounds may be effective in targeting and accumulating in solid tumors, and may also be useful in the treatment of HIV and other viruses. The conjugates provided herein may also be used to treat platinum- and paclitaxel-resistant tumors.

Methods of treatment comprise administering to a mammal in need thereof a therapeutically effective amount of a composition or formulation containing a topotecan polymer conjugate as described herein.

A therapeutically effective dosage amount will vary from conjugate to conjugate, patient to patient, and will depend upon factors such as the condition of the patient, the activity of the particular active agent employed, and the route of delivery. Dosages from about 0.5 to about 100 mg topotecan/m² body weight, preferably from about 1.0 to about 10 mg/m², are preferred. The dosage amount may be reduced, if necessary, to manage myelosuppression during treatment. Administration of growth factors such as G-SCF amd GM-CSF may also be used to manage associated hematologic toxicities, e.g., to manage neutropenia and prevent neutropenic fevers and infection. When administered conjointly with other pharmaceutically active agents, even less of the topotecan conjugate may be therapeutically effective. The range set above is illustrative and those skilled in the art will determine optimal dosing of the topotecan conjugate based on clinical experience and the particular treatment indication. The dosing schedule will vary depending upon mode of administration, particular indication being treated, patient considerations, and the like. Representative dosing schedules include daily dosing on days 1-5 of a 21 day course of treatment, as well as administration on days 1-3 of a 21 day course of treatment. Additional treatment regimens include 3-day infusion and weekly infusion schedules, and the like.

Methods of treatment also include administering a therapeutically effective amount of a topotecan conjugate as provided herein in conjunction with a second anticancer or other active agent, such as cisplatin, docetaxel, cyclophosphamide, phenoxodiol, lapatinib, and the like. For example, in the treatment of colorectal cancer, a topotecan polymer conjugate compound may be administered in conjunction with chemotherapeutics such as 5-fluorouracil or leucovorin xeloda, or with agents such as avastin, Erbitux® (cetuximab), or Vectibix™ (panitumumab). In the treatment of breast cancer, therapy may include administration of a topotecan polymer conjugate as described herein, optionally in combination with xeloda, paclitaxel, docetaxel, or abraxane. In treating lung cancer, therapy may include, along with administration of a topotecan polymer conjugate, administration of cis-platin, carboplatin, gemcitabine, alimpta, and docetaxel.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All articles, books, patents and other publications referenced herein are hereby incorporated by reference in their entireties.

### EXAMPLES

The practice of the invention will employ, unless otherwise indicated, conventional techniques of organic synthesis and the like, which are within the skill of the art. Such techniques are fully explained in the literature. Reagents and materials are commercially available unless specifically stated to the contrary. See, for example, J. March, Advanced Organic Chemistry: Reactions Mechanisms and Structure, 4th Ed. (New York: Wiley-Interscience, 1992), supra, and Comprehensive Organic Functional Group Transformations II, Volumes 1-7, Second Ed.: A Comprehensive Review of the Synthetic Literature 1995-2003 (Organic Chemistry Series), Eds. Katritsky, A. R., et al., Elsevier Science.

In the following examples, efforts have been made to ensure accuracy with respect to numbers used (e.g., amounts, temperatures, etc.) but some experimental error and deviation should be accounted for. Unless indicated otherwise, temperature is in degrees C. and pressure is at or near atmospheric pressure at sea level.

The following examples illustrate certain aspects and advantages of the present invention, however, the present invention is in no way considered to be limited to the particular embodiments described below. The definition for abbreviations used herein are provided below.

Ar: argon
CM: carboxymethyl or carboxymethylene (—CH₂COOH)
DCC: 1,3-dicyclohexylcarbodiimide
DCM: dichloromethane

### DMAP: 4-(N,N-dimethylamino)pyridine

GLY: glycine
HCl hydrochloric acid
RP-HPLC: reverse-phase high performance liquid chromatography
IPA: isopropyl alcohol
IRT: irinotecan
IPC: ion pair chromatography
MeOH: methanol
MTBE: methyl tert-butyl ether
MW: molecular weight
NMR: nuclear magnetic resonance
PEG: polyethylene glycol
RT: room temperature
SCM: succinimidylcarboxymethyl (—CH₂—COO-N-succinimidyl)
TEA: triethylamine
TFA: trifluoroacetic acid
THF: tetrahydrofuran

Materials and Methods

Topotecan hydrochloride was purchased from ChemWerth USA (Woodbridge, Conn.).

4-Arm-PEG_{20K}-SCM was prepared from 4-arm-PEG_{20K}-OH. 4-arm-PEG₂ₘ-OH, linear mPEG₂₀ₖ-SCM and dumbbell PEG₂₀ₖ-SCM were obtained were obtained from NOF America Corporation (White Plains, N.Y.) or from ChemOrganics (Houston, Tex.).

Sources of the following reagents were as follows: Glycine tert-butyl ester (98%, Aldrich); 4-dimethylaminopyridine (DMAP, 99%, Aldrich); N,N′-diisopropylcarbodiimide (DIC, 99%, Acros), N,N′-dicyclohexylcarbodiimide (DCC, 99%, Acros), N,N-diisopropylethylamine (DIPEA, 99%, Aldrich), and p-toluenesulfonic acid (PTSA, 98.5%, Aldrich), and all reagents were used as received. Solvents were dried before use.

All ¹HNMR data was generated by a 300 or 400 MHz NMR spectrometer manufactured by Bruker.

### Example 1

### Synthesis of Various Poly(ethylene glycol)20kD-Carboxy Methyl-Glycine Linked Topotecan Conjugates

Various PEG-carboxymethyl (CM)-glycine linked topotecan conjugates were synthesized, each having a different PEG architecture (linear, dumbbell, and 4-arm pentaerythritolyl-core). The yields at each step typically were in a range from about 80%-90%. Structures of the resulting conjugates were as follows:

### Linear:

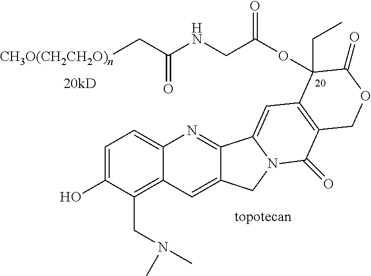

Shorthand: “mPEG20k-CM-GLY-O-20-Top” or “mPEG-topotecan”

### Dumbbell:

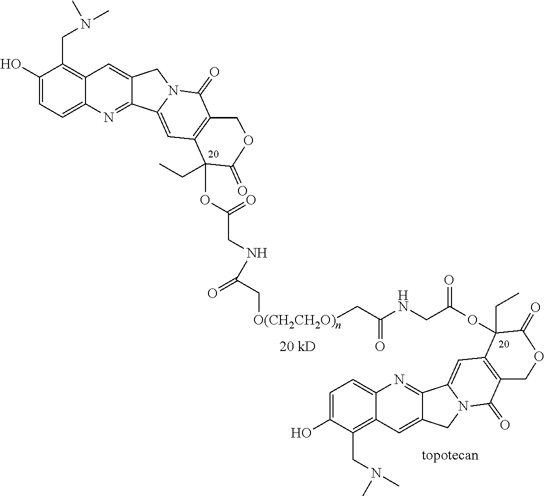

Shorthand: Dumbbell PEG20k-(CM-GLY-O-20-Top)₂ or “dumbbell-PEG topotecan”

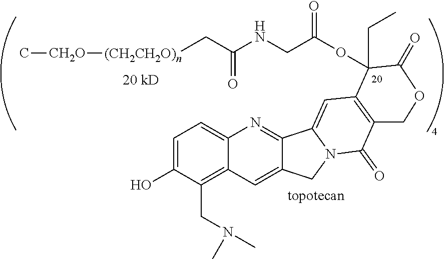

### 4-Arm:

Shorthand: “4-arm-PEG20k-CM-GLY-O-20-Top” or “4-arm PEG Topotecan”

General Synthesis:

Each of the conjugates shown above was generally synthesized according to the following reaction scheme, where the particular architecture of the polymer reagent differed according to the final product formed (i.e., linear, dumbbell, 4-arm) as illustrated above.

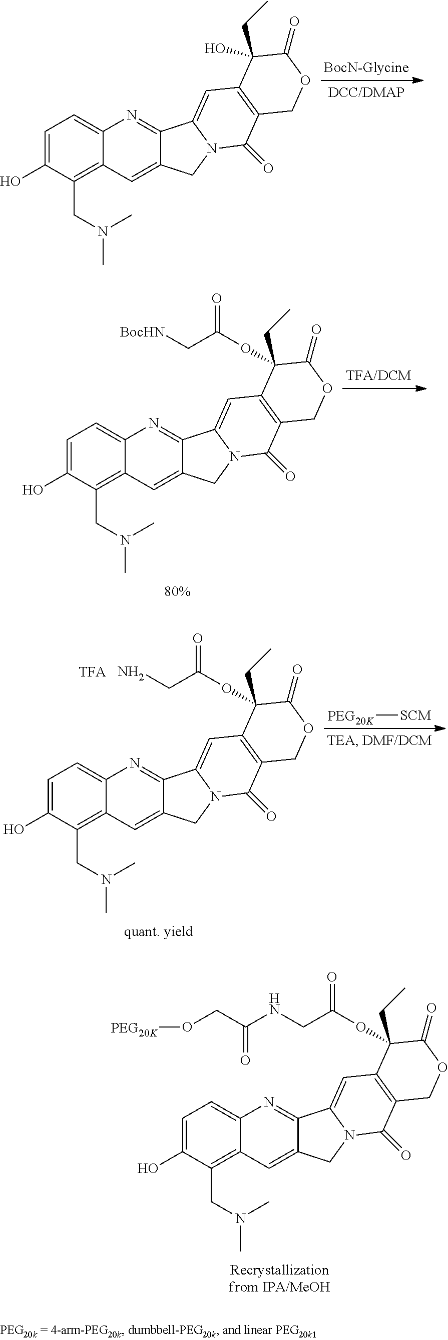

### A. Synthesis of t-Boc-Glycine-Topotecan

In a flask, topotecan (0.1704 mmoles), t-Boc-Glycine (0.3408 mmoles), and DMAP (0.1704 mmoles) were dissolved in anhydrous dichloromethane (DCM). To the solution was added DCC (0.3408 mmoles) dissolved in anhydrous DCM. The solution was stirred overnight at room temperature. The solid was removed through a coarse frit, and the solution was washed with 0.1N HCl in a separatory funnel. The organic phase was further washed with deionized H₂O in a separatory funnel and then dried with Na₂SO₄. The solvent was removed using rotary evaporation and the product was further dried under vacuum.

### B. Deprotection of t-Boc-Glycine-Topotecan

0.1 g t-Boc-Glycine-topotecan (0.137 mmoles) was dissolved in anhydrous DCM. To the solution was added trifluoroacetic acid (TFA, 6.85 mmoles). The solution was stirred at room temperature under argon for 1 hour. The solvent was removed using rotary evaporation. The crude product was dissolved in 0.1 mL MeOH and then precipitated in 25 mL of ether. The suspension was stirred in an ice bath for approximately 30 minutes. The product, a pale to dark yellow solid, was collected by filtration and dried under vacuum.

### C. Conjugate Preparation—Covalent Attachment to Activated CM-Poly(ethylene glycol)

The TFA salt of glycine-topotecan was then conjugated to 4-arm poly(ethylene glycol) MW 20,000 Da succinimidyl acetate in the presence of triethyl amine (TEA) at room temperature and under nitrogen in a cosolvent of DCM/DMF for 24 to 26 hours. The conjugate was precipitated by the addition of methyl tert-butyl ether (MTBE) or isopropyl alcohol (IPA) and isolated by filtration. The product was purified through a series of successive precipitations from isopropyl alcohol/methanol cosolvent. Finally, the purified product was isolated by filtration and dried under vacuum at 25° C. The structure of the product was further confirmed by ¹³C-NMR, FTIR, and MALDI.

Regarding the 4-arm compound, although the structure provided above depicts 4-arm PEG-topotecan as having a discrete molecular weight and complete polymer loading, i.e., a topotecan molecule attached to each arm of the 4-arm polymer core, more plausibly, upon reaction, the polymer (having a particular molecular weight distribution) produces a compound having an average of 3.6 molecules of topotecan per 4-armed polymer core (equal to a drug loading of 6.9 percent). Dumbbell PEG topotecan was calculated to possess, on average, a topotecan loading value of 1.7 topotecan molecules per polymer core (equal to a drug loading of 3.4 percent).

Purity of the product, based upon analyses of different product lots, was about 97% for 4-arm PEG topotecan and about 96% for dumbbell PEG topotecan. Free topotecan in the final product was typically 1% or lower.

### Example 2

### Hydrolysis Studies

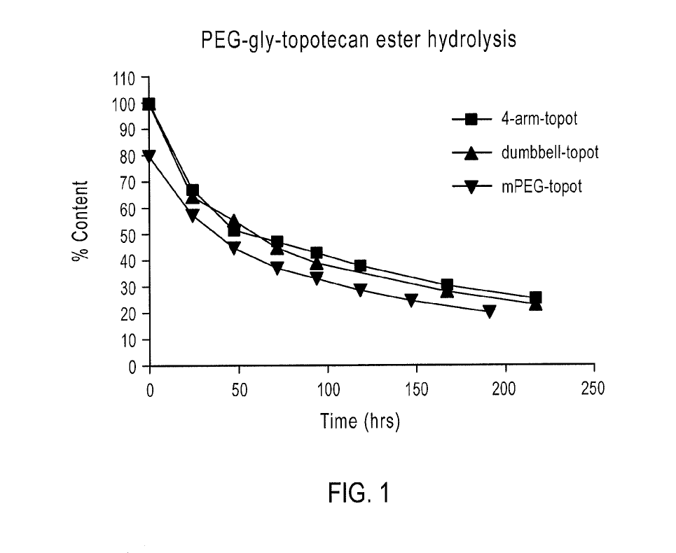

Hydrolysis experiments were carried out on each of the PEG-topotecan conjugates (linear, dumbbell, 4-armed). The studies were performed in phosphate-buffered saline (PBS) buffer at pH 7.2 and 37° C. The results are shown in FIG. 1. The hydrolysis profiles of each of the conjugates were similar, although the 4-arm PEG topotecan conjugate and the dumbbell structures appear to be slightly more resistant to hydrolysis than their linear counterpart, mPEG-topotecan.

The hydrolysis routes for PEG-topotecan conjugates include release of free drug, topotecan, via hydrolysis of the ester bond, as well as hydrolysis and opening of the topotecan lactone as illustrated below.

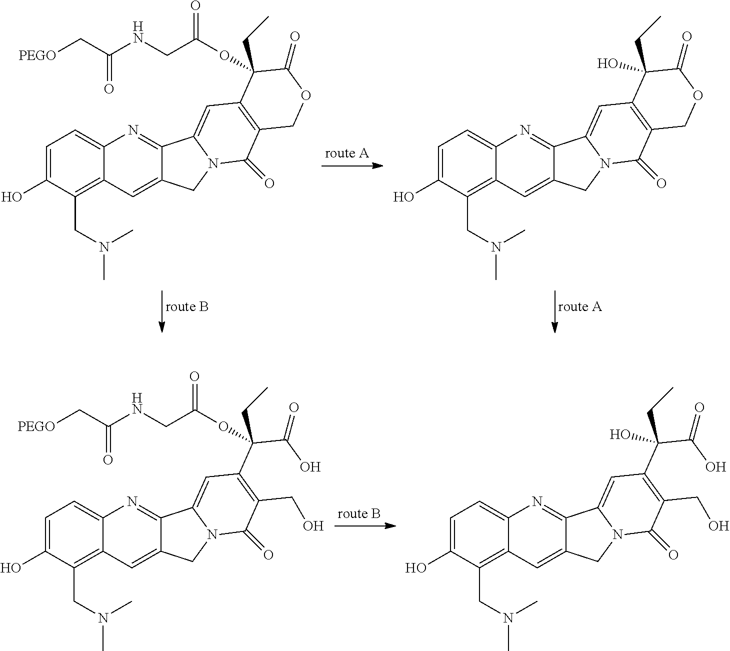

In examining the hydrolytic stability (i.e., ring opening) of the topotecan lactone ring, there was little difference in the rate of ring opening under the hydrolysis conditions examined for unconjugated topotecan versus dumbbell-PEG topotecan (data/graph not shown).

### Example 3

### Maximum Tolerated Dose Studies in Female Athymic Nude Mice

A study was conducted to evaluate the tolerance of female athymic NCr-nu/nu mice to treatment with 4-arm PEG topotecan, dumbbell PEG-topotecan, and topotecan when administered intravenously.

Animal Care:

Six-weeks-old female athymic NCr-nu/nu mice were purchased from Charles River Laboratories, Inc. (Wilmington, Mass.) and acclimated in the laboratories for 9 days prior to experimentation. The mice were housed in microisolator cages with up to five animals per cage. Each animal in the cage was identified by an ear punch. All mice were on a 12-hour light/dark cycle and received filtered water and sterilizable rodent diet (Harlan-Teklad TD8656) ad libitum. Cages were changed twice weekly. The animals were observed daily and clinical signs were noted. The day on which the study commenced was designated as Day 1.

Drug Formulation:

4-arm PEG topotecan was prepared at a concentration of 0.5 mglmL solution of topotecan on each day of treatment by dissolving 15.15 mg of the compound per mL of saline. The lower dosages were achieved by further dilutions with saline. The dosing solutions were treated as light-sensitive (formulated under amber lights and dispensed into brown bottles), stored at room temperature, and were administered within 2 hours of formulation. Dumbbell PEG-topotecan was prepared at a concentration of 1.0 mglmL solution of topotecan on each day of treatment by dissolving 42.37 mg of the compound per mL of saline. The lower dosages were achieved by further dilutions with saline. The dosing solutions were treated as light-sensitive (formulated under amber lights and dispensed into brown bottles), stored at room temperature, and were administered within 2 h of formulation. Topotecan was prepared at a concentration of 0.75 mglmL on each day of treatment in saline. The lower dosages were achieved by further dilutions with saline. The dosing solutions were treated as light-sensitive (formulated under amber lights and dispensed into brown bottles), were kept on ice, and were administered within 30 min of formulation. Saline was obtained from Phoenix Pharmaceutical, Inc.

Treatment:

The study consisted of nine treatment groups of five animals each for a total of 45 mice at the start of treatment on Day 1. 4-arm PEG topotecan at dosages of 10, 5, and 2.5 mg/kg/injection; dumbbell PEG topotecan at dosages of 20, 15, and 10 mg/kg/inj, and topotecan at dosages of 15, 10, and 5 mg/kg/inj were administered intravenously once every four days for a total of three injections (Q4D×3) beginning on Day 1 (i.e., Days 1, 5, and 9). All treatment was administered by exact body weight using an injection volume of 0.2 mL/10 g of body weight.

Mortality and Body Weights:

The animals were checked daily and mortality was recorded. The animals were weighed twice weekly starting with the first day of treatment, Day 1.

Parameters Evaluated:

The parameters evaluated included the number of 21-day survivors. Recorded were the summary of the test article, individual body weights, mean body weights, and the change in mean body weight relative to the mean body weight on the first day of collection (in grams and as a percent), and mortality.

Results:

Tolerance of non-tumor-bearing female athymic NCr-nu/nu mice to treatment with 4-arm PEG topotecan at dosages of 10, 5, and 2.5 mg/kg/inj, dumbbell PEG topotecan at dosages of 20, 15, and 10 mg/kg/inj, and topotecan at dosages of 15, 10, and 5 mg/kg/inj administered iv was tested on a Q4D×3 treatment schedule beginning on Day 1. The treatment with 4-arm PEG topotecan at dosages of 10, 5, and 2.5 mg/kg/inj resulted in no deaths. There was a 21% (4.8 g) maximum average body weight loss at a dosage of 10 mg/kg/inj, a 1% (0.2 g) maximum average body weight loss at a dosage of 5 mg/kg/inj, and a 5% (1.2 g) maximum average body weight loss at a dosage of 2.5 mg/kg/inj. The treatment with dumbbell PEG topotecan at dosages of 20, 15, and 10 mg/kg/inj resulted in no deaths. There were maximum losses in average body weight of 9% (2.0 g), 2% (0.5 g), and 0%, respectively, for dosages of 20, 15, and 10 mg/kg/inj. The treatment with topotecan at dosages of 15, 10, and 5 mg/kg/inj resulted in no deaths. There were maximum losses in average body weight of 4% (1.0 g), 0%, and 0%, respectively, for dosages of 15, 10, and 5 mg/kg/inj.

Conclusion:

Non-tumor-bearing female athymic NCr-nu/nu mice were able to tolerate iv treatment with 4-arm PEG topotecan at dosages of 10, 5, and 2.5 mg/kg/inj, dumbbell PEG-topotecan at dosages of 20, 15, and 10 mg/kg/inj, and topotecan at dosages of 15, 10, and 5 mg/kg/inj without deaths when administered on a Q4D×3 treatment schedule; however, treatment with 4-arm PEG topotecan at a dosage of 10 mg/kg/inj produced a 21% maximum body weight loss. The maximum tolerated dosages [MTD, defined as the dosage which does not result in more than 10% deaths of animals (one death out of ten animals) or produces no more than 20% average body weight loss] for 4-arm PEG topotecan, dumbbell PEG topotecan, and topotecan when administered iv on a Q4D×3 treatment schedule were 5, 20, and 15 mg/kg/inj, respectively, in this study.

### Example 4

### HT29 Colorectal Cancer Model: Efficacy of Exemplary Topotecan Conjugates in HT29 Xenograft Study in Female Athymic Nude Mice

The study was undertaken to evaluate the antitumor efficacy of 4-arm PEG-topotecan, dumbbell PEG topotecan, and topotecan when administered intravenously (iv) against subcutaneously (sc)-implanted HT29 human colon tumor in female athymic NCr-nu/nu mice.

Animal Care:

Six-weeks-old female athymic NCr-nu/nu mice were purchased from Charles River Laboratories, Inc. (Wilmington, Mass.) and acclimated in the laboratories for nine days prior to experimentation. The mice were housed in microisolator cages, up to five animals per cage. Each animal in the cage was identified by an ear punch. All mice were on a 12-hour light/dark cycle and received filtered municipal water and sterilizable rodent diet (Harlan-Teklad TD8656) ad libitum. Cages were changed twice weekly. The animals were observed daily and clinical signs were noted.

Tumor Model.

Thirty-to-forty mg fragments of HT29 human colon tumor, propagated in an in vivo passage, were implanted sc on the right flank area of mice using a12-gauge trocar needle. The day of tumor implantation was designated as Day 0. Tumors were allowed to reach 100-245 mg in weight (100-245 mm³ in size) before the start of treatment. A sufficient number of mice were implanted so that animals with tumors in a weight range as narrow as possible were selected for the trial on the day of treatment initiation (Day 14 after tumor implantation). Those animals selected with tumors in the proper size range on Day 14 were placed in several large cages and then assigned randomly to the various treatment groups. A few animals were switched between groups so that the median tumor weight range on the first day of treatment was as narrow as possible (167-188 mg).

Drug Formulation:

The compound, 4-arm PEG topotecan, was prepared at a concentration of 0.375 mg/mL solution of topotecan on each day of treatment by dissolving 11.36 mg of the compound per mL of saline. The lower dosage was achieved by further dilution with saline. The dosing solutions were treated as light-sensitive (formulated under amber lights and dispensed into brown bottles), stored at room temperature, and administered within 2 h of formulation. Dumbbell-PEG topotecan was prepared at a concentration of 1.0 mg/mL solution of topotecan on each day of treatment by dissolving 42.37 mg of the compound per mL of saline. The lower dosage was achieved by further dilution with saline. The dosing solutions were treated as light-sensitive (formulated under amber lights and dispensed into brown bottles), were stored at room temperature, and were administered within 2 h of formulation. Topotecan was prepared at a concentration of 1.0 mg/mL on each day of treatment in saline. The lower dosages were achieved by further dilutions with saline. The dosing solutions were treated as light-sensitive (formulated under amber lights and dispensed into brown bottles), were kept on ice, and were administered within 30 min of formulation. Saline was obtained from Phoenix Pharmaceutical, Inc.

Drug Treatment:

The study consisted of a vehicle-treated control group and seven treatment groups with ten mice each for a total of 80 mice at the start of treatment. 4-arm-topotecan (at dosages of 7.5 and 5.0 mg/kg/inj), dumbbell PEG topotecan (at dosages of 20 and 15 mg/kg/inj), and topotecan (at dosages of 20, 15, and 7.5 mg/kg/inj) were each administered intravenously once every four days for a total of three injections (depicted as Q4D×3(14); i.e., Days 14, 18, and 22). The control group was treated intravenously with saline on the same treatment schedule. All treatments were administered by exact body weight with the injection volume being 0.2 mL/10 g of body weight.

Tumor Measurements and Body Weights:

Tumors were measured (the limit of detection being 32 mg (4×4 mm)) and the animals were weighed twice weekly starting with the first day of treatment. Tumor volume was determined by caliper measurements (mm) and using the formula for an ellipsoid sphere: L×W²/2=mm³, where L and W refer to the larger and smaller perpendicular dimensions collected at each measurement. This formula is also used to calculate tumor weight, assuming unit density (1 mm³=1 mg). Individual tumor and body weight measurements were collected.

Parameters Evaluated:

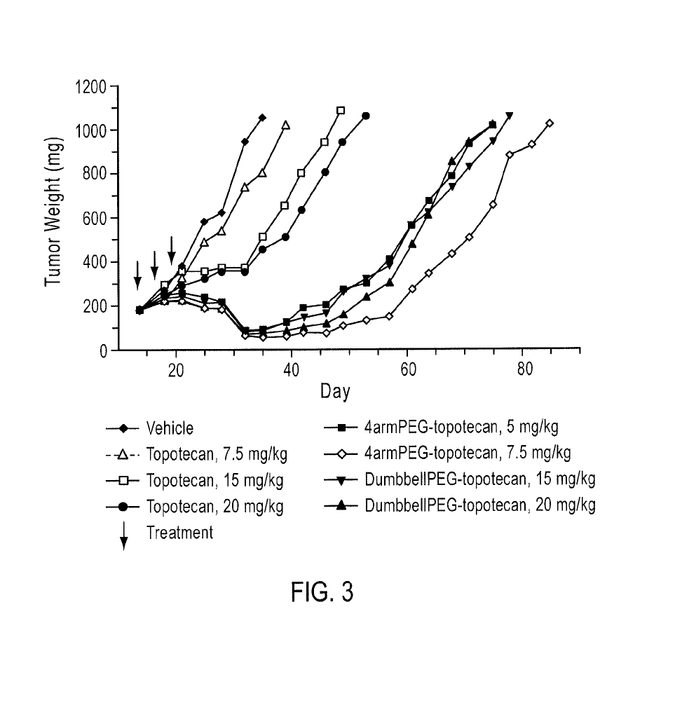

Number of non-specific deaths, number of complete tumor regressions, number of tumor-free survivors on Day 99, and median number of days for the tumors in each group to reach 1,000 mg were calculated. The median time (in days) to reach 1,000 mg was used in the calculation of the overall delay in the growth of the median tumor (T-C; T=treated group; C=control group). Results are summarized in FIG. 3. Individual tumor weights, group median tumor weights, TIC (%) based on median tumor weights, group mean tumor weights, standard error of the mean tumor weights, the time for the individual animal's tumor to reach 1,000 mg, individual body weights, mean body weights, and the change in mean body weights relative to the mean body weight on the first day of collection (in grams and as a percent) were collected.

Statistical Analysis:

The time for the individual animal's tumor to reach 1,000 mg was used as the endpoint in either a Student's t-test or a life table analysis (stratified Kaplan-Meier estimation followed by the Mantel-Haenszellog-rank test) in order to statistically compare the growth data between groups. A life table analysis allows one to compare the growth data between the groups using the animals whose tumors did not reach the evaluation point, by censoring them.

Results:

Tumors of all ten mice bearing sc implanted HT29 human colon tumor in the vehicle-treated control group grew progressively, with the median tumor reaching the evaluation point of 1,000 mg in 34.1 days. All surviving animals whose tumor weight was greater than or equal to 1 g on Day 61 were to be euthanized; thereafter, any animal whose tumor weight reached 1 g was euthanized. Accordingly, animals 1-6 and 8-10 in Group 1 were euthanized on Day 61. Treatment with vehicle (saline) administered iv on a Q4D×3 treatment schedule resulted in a maximum loss in average body weight of 6% (1.5 g).

Treatment with 4-arm PEG topotecan at dosages of 7.5 and 5.0 mg/kg/inj administered iv on a Q4D×3 treatment schedule resulted in median times to 1,000 mg of 82.9 and 71.0 days, which corresponded to tumor growth delay (T-C) values of 48.8 and 36.9 days, respectively. There was one late death in the group treated with a dosage of 5.0 mg/kg/inj (Group 3, animal 10 on Day 85), with its tumor not having reached the evaluation point. The treatment with 4-arm PEG topotecan resulted in maximum losses in average body weight of 9% (2.2 g) and 5% (1.3 g), respectively, for dosages of 7.5 and 5.0 mg/kg/inj.

Treatment with dumbbell PEG topotecan at dosages of 20 and 15 mg/kg/inj administered iv on a Q4D×3 treatment schedule resulted in median times to 1,000 mg of 72.1 and 75.2 days, which corresponded to T-C values of 38.0 and 41.1 days, respectively. A dosage of 20 mg/kg/inj resulted in one tumor-free survivor (Group 4, animal 3). The treatment with dumbbell PEG topotecan resulted in maximum losses in average body weight of 13% (3.0 g) and 7% (1.8 g), respectively, for dosages of 20 and 15 mg/kg/inj.

Treatment with topotecan at dosages of 20, 15, and 7.5 mg/kg/inj administered iv on a Q4D×3 treatment schedule resulted in median times to 1,000 mg of 49.9, 46.8, and 38.5 days, which corresponded to T-C values of 15.8, 12.7, and 4.4 days, respectively. A dosage of 20 mg/kg/inj resulted in the deaths of two animals (Group 6, animal 8 on Day 21 and animal 10 on Day 25). A dosage of 15 mg/kg/inj resulted in the deaths of two animals (Group, animal 8 on Day 21 and animal 10 on Day 25). The treatment with topotecan resulted in maximum losses in average body weight of 15% (3.5 g), 11% (2.6 g), and 6% (1.3 g), respectively, for dosages of 20, 15, and 7.5 mg/kg/inj. All surviving animals treated with topotecan were euthanized on Day 61.

A statistical analysis was conducted to compare treatment with one compound to treatment with another compound. The difference between the groups was considered to be significant if the p value was equal to or less than 0.05.

A statistically significant difference was not observed when the individual times for the animal's tumor to reach 1,000 mg for a dosage of 7.5 mg/kg/inj of 4-arm PEG topotecan were compared to those for a dosage of 20 mg/kg/inj of dumbbell PEG topotecan (p value of 0.540) nor when the individual times for the animal's tumor to reach 1,000 mg for a dosage of 5.0 mg/kg/inj of 4-arm PEG topotecan were compared to those for a dosage of 15 mg/kg/inj of dumbbell PEG topotecan (p value of 0.552).

Statistically significant differences were observed for 4-arm PEG topotecan when the individual times for the animal's tumor to reach 1,000 mg for a dosage of 7.5 mg/kg/inj of 4-arm PEG topotecan were compared to those for a dosage of 20 mg/kg/inj of topotecan (p value of <0.001) or when the individual times for the animal's tumor to reach 1,000 mg for a dosage of 5.0 mg/kg/inj of 4-arm PEG topotecan were compared to those for a dosage of 15 mg/kg/inj of topotecan (p value of <0.001).

Statistically significant differences were observed for dumbbell PEG topotecan when the individual times for the animal's tumor to reach 1,000 mg for a dosage of 20 mg/kg/inj of dumbbell PEG topotecan were compared to those for a dosage of 20 mg/kg/inj of topotecan (p value of <0.001) or when the individual times for the animal's tumor to reach 1,000 mg for a dosage of 15 mg/kg/inj of dumbbell PEG topotecan were compared to those for a dosage of 15 mg/kg/inj of topotecan (p value of <0.001).

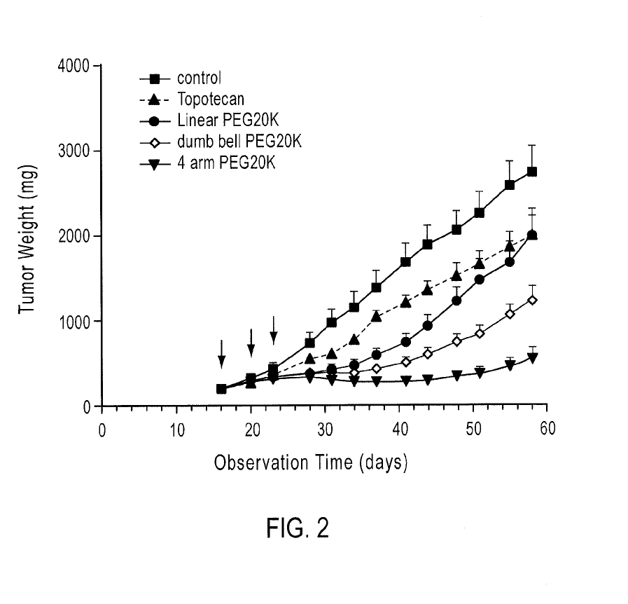

A graphical presentation of the response of sc implanted HT29 human colon tumor to treatment with 4-arm PEG topotecan, dumbbell PEG topotecan, and topotecan may be seen in FIG. 3. The results in FIG. 2 are from an earlier study (not described in detail herein, but upon which the current example design was based). The graph is included herein to demonstrate the superior nature of the 4-arm- and dumbbell topotecan conjugates in comparison to mPEG topotecan.

Summary:

Treatment with two exemplary PEG topotecan conjugates, 4-arm PEG topotecan and dumbbell PEG topotecan, along with topotecan per se, administered iv on a Q4D×3 treatment schedule, was carried out. The compounds were tested for their ability to inhibit the growth of HT29 human colon tumor xenografts implanted sc in female athymic NCr-nu/nu mice. The growth of HT29 human colon tumor xenografts was inhibited significantly when treated with 4-arm PEG topotecan at dosages of 7.5 and 5.0 mg/kg/inj, producing growth delay values of 48.8 and 36.9 days, respectively. The growth of HT29 human colon tumor xenografts was also inhibited significantly when treated with dumbbell PEG topotecan at dosages of 20 and 15 mg/kg/inj, producing growth delay values of 38.0 and 41.1 days, respectively. Dosages of 20 and 15 mg/kg/inj of topotecan were effective in inhibiting the growth of HT29 human colon tumor xenografts, whereas a dosage of 7.5 mg/kg/inj was minimally effective, producing growth delay values of 15.8, 12.7, and 4.4 days, respectively. Statistically significant differences were observed for 4-arm PEG topotecan at dosages of 7.5 and 5.0 mg/kg/inj when compared to topotecan at dosages of 20 and 15, respectively (p values of <0.001 and <0.001); however, no significant differences were observed when the individual times for the animal's tumor to reach 1,000 mg for dosages of 7.5 and 5.0 mg/kg/inj of 4-arm PEG topotecan were compared to those for dosages of 20 and 15 mg/kg/inj of dumbbell PEG topotecan, respectively (pvalues of 0.540 and 0.552). Statistically significant differences were observed for dumbbell PEG topotecan at dosages of 20 and 15 mg/kg/inj when compared to topotecan at dosages of 20 and 15 mg/kg/inj, respectively (p values of <0.001 and <0.001).

Based upon the above results, it can be seen that both the 4-arm and 2-arm (dumbbell) PEG-topotecan architectures exhibit superior efficacy in comparison to unconjugated topotecan. Tumor growth suppression was observed at levels below the maximum-tolerated-dose (MTD), and no deaths were observed in either the 4-arm PEG topotecan or the dumbbell PEG topotecan groups. In contrast, two deaths were occurred in each of the 15 mg/kg and 20 mg/kg topotecan groups.

TABLE 2
Summary of Results

| | | | | | | Median | |
|---|---|---|---|---|---|---|---|
| | | | Non- | Regres- | Tumor | Days to | |
| | Com- | | Specific | sions | Free on | 1000 | Growth |
| Group | pound | Dose | Deaths | (100%) | Day 99 | mg | Delay |
| 1 | Con- | 0 | 0 | 0 | 0 | 34.1 | NA |
| | trol | | | | | | |
| 2 | 4-APT | 7.5 | 0 | 0 | 0 | 82.9 | 48.8 |
| 3 | 4-APT | 5.0 | 0 | 0 | 0 | 71.0 | 36.9 |
| 4 | DPT | 20.0 | 0 | 1 | 1 | 72.1 | 38.0 |
| 5 | DPT | 15.0 | 0 | 0 | 0 | 75.2 | 41.1 |
| 6 | Top | 20.0 | 2 | 0 | 0 | 49.9 | 15.8 |
| 7 | Top | 15.0 | 2 | 0 | 0 | 46.8 | 12.7 |
| 8 | Top | 7.5 | 0 | 0 | 0 | 38.5 | 4.4 |

*Each group contained 10 animals
**Compound Abbreviations; “4-APT” corresponds to 4-arm PEG Topotecan; “DPT” corresponds to dumbbell PEG topotecan; and “Top” corresponds to topotecan
***Dosages are in mg/kg/inj; route of administration is intravenous; dosing schedule: Q4Dx3(14)
****Growth delay calculations are based on median days to 1000 mg

### Example 5

### H460 Lung Cancer Model: Efficacy of Exemplary Topotecan Conjugates in H460 Xenograft Study in Female Athymic Nude Mice

The purpose of the study was to evaluate the antitumor efficacy of exemplary PEG topotecan conjugates along with unmodified topotecan when administered against subcutaneously (sc) implanted human NCI-H460 lung tumor xenografts in male athymic nude mice.

Animal Care:

Six-weeks-old male athymic nude mice were purchased from Charles River (Wilmington, Mass.) and acclimated in the laboratories prior to experimentation. The animals were housed in microisolator cages, up to five per cage in a 12-hour light/dark cycle. The animals received filtered municipal water and sterilizable rodent diet (Harlan-Teklad TD8656) ad libitum. Cages were changed twice weekly. The animals were observed daily and clinical signs were noted.

Tumor Model:

Thirty-to-forty mg fragments of human NCI-H460 lung tumor were implanted sc in mice near the right axillary area using a 12-gauge trocar needle and allowed to grow. The day of tumor implantation was designated as day 0. Tumors were allowed to reach 100-234 mg in weight (100-234 mm³ in size) before the start of treatment. A sufficient number of mice was implanted so that tumors in a weight range as narrow as possible were selected for the trial on the day of treatment initiation (day 7 after tumor implantation). Those animals selected with tumors in the proper size range were assigned to the various treatment groups so that the median tumor weights on the first day of treatment were as close to each other as possible (137-154 mg).

Drug Formulation:

On each day of injection, a 15.625 mg/mL solution of PEG-topotecan (the amount of PEG-topotecan being 31.25 times the topotecan dosage) was prepared in saline. A portion of this solution was diluted with saline to the lower concentrations. All three solutions were quite viscous. A 0.5 mg/mL solution of topotecan was prepared in saline on each day of treatment. Lower dosages were achieved by diluting this solution with saline. All dosing solutions were administered by exact body weight using an injection volume of 0.2 mL/10 g of body weight. Both PEG-topotecan and topotecan solutions were prepared in subdued light with PEG-topotecan injected within two hours of preparation and topotecan injected within 30 minutes of preparation.

Drug Treatment:

The experiment consisted of six treatment groups of ten mice per group and one vehicle-treated control group also with ten mice for a total of 70 mice on the first day of treatment. Both PEG-topotecan and topotecan were administered intravenously (iv) as three injections given four days apart (q4d×3) at dosages of 10, 2.5, and 1 mg/kg/dose. The control group (group 1) was treated with the vehicle (saline), which was also administered iv q4d×3.

Tumor Measurements and Body Weights:

The sc tumors were measured and the animals were weighed twice weekly starting with the first day of treatment. Tumor volume was determined by caliper measurements (mm) and using the formula for an ellipsoid sphere: Lx W²/2=mm³, where Land W refer to the larger and smaller perpendicular dimensions collected at each measurement. This formula is also used to calculate tumor weight, assuming unit density (1 mm³=1 mg).

Study Duration:

The study was terminated 55 days after tumor implantation. Any animal whose tumor became ulcerated or reached 4,000 mg was euthanized prior to study termination.

Parameters Evaluated:

Number of non-specific deaths, number of partial and complete tumor regressions, number of tumor-free survivors, and the individual animals' times to reach three tumor mass doublings were determined. The median time to reach three tumor mass doublings in the treatment groups (T) and control group (C) was used in the calculation of the overall delay in the growth of the median tumor (T-C).

Statistical Analysis:

The individual animal's time to reach three tumor mass doublings was used as the endpoint in a Student's t-test (or Mann-Whitney rank sum test) in order to statistically compare the growth data in all groups except for group 10. The nonparametric test was used when the data set did not pass the normality or equal variance test.

Results:

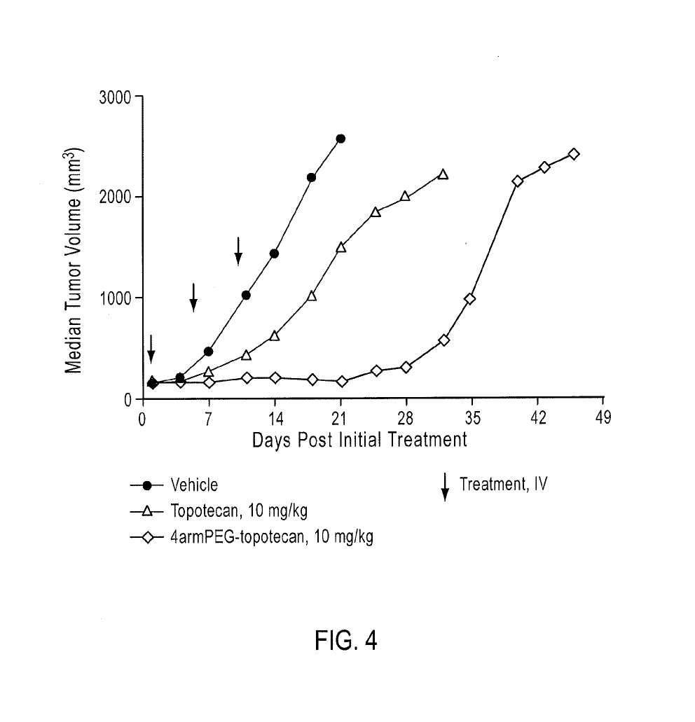

Tumors in the vehicle-treated control group grew well in all 10 mice. The median tumor reached three tumor mass doublings in 11.2 days. Administration of PEG-topotecan at dosages of 10, 2.5, and 1.0 mg/kg/dose was tolerated without deaths but with a maximum average weight loss of 21% (6 g), 10% (3 g), and 7% (2 g) in each group, respectively. The lost weight was regained following cessation of treatment in all groups. Median tumor growth delays were 24.5, 8.7, and 1.4 days, respectively, for the dosages of 10, 2.5, 1.0 mglkg/dose. Treatment with topotecan at a dosage of 10 mglkg/dose resulted in one early death which occurred on day 13. There was also an 11% (3 g) loss in mean body weight associated with this treatment. Treatment with the lower dosages of 2.5 and 1.0 mglkg/dose was tolerated without deaths. The average losses in mean body weight for the dosages of 2.5 and 1.0 mg/kg/dose were 7% (2 g) and 11% (3 g), respectively. Lost weight was regained in all three topotecan-treated groups following cessation of treatment. The response of the NCI-H460 lung tumor to treatment with PEG-topotecan or topotecan is shown graphically in FIG. 4.

Summary:

Growth delays elicited by treatment with PEG-topotecan at dosages of 10 and 2.5 mg/kg/dose were significantly greater than those elicited by treatment with topotecan at the same dosages. At the lowest dosage tested, 1.0 mg/kg/dose, the growth delay resulting from treatment with topotecan was significantly greater than that resulting from treatment with PEG-topotecan at a dosage of 1.0 mg/kg/dose.

### Example 6

### Pharmacokinetic Analysis of Exemplary Topotecan Conjugates Following a Single IV Infusion in Beagle Dogs

The plasma half-lives of exemplary PEG-topotecan conjugates (4-arm PEG topotecan, dumbbell PEG topotecan, linear mPEG topotecan were determined as follows.

Administration.

All animals were fasted overnight prior to dosing and through the first 4 hours of blood sample collection (total fasting time not to exceed 24 hours). Each animal in Groups 1-4 received a single intravenous (IV) dose of vehicle, topotecan, 4-arm-PEG topotecan, or dumbbell PEG topotecan at a dose of 4 mg/kg/hr, where for the topotecan equivalent, a total dose of 2 mg/kg/ was administered for a 0.5 hour infusion. Intravenous doses were administered via the cephalic (or other suitable) vein. The dose was administered as an intravenous infusion over approximately 30 minutes at 4 mL/kg/hr, with all blood collection times calculated from the start of dosing, if applicable. Blood samples were collected predose and at 0.25 (15 min.), 29 min (just prior to end of infusion), 1, 2, 4, 6, 8, 12, 24, 48, 72, 96, 120, 168, 216, 264, 312, and 336 hours post-dose. Urine samples were collected predose and at 0-24, 24-48, 48-72, and 72-96 hours post-dose.

Phamiacokinetic Blood Collection:

Blood samples (approximately 2 mL/sample) were collected from the jugular vein at the time points specified above and placed into tubes containing sodium fluoride/Na₂EDTA. All blood samples were placed on an ice block (or wet ice) following collection. The samples were centrifuged within 30 minutes of collection and the resulting plasma was separated. Two 500 μL aliquots of plasma were transferred into tubes. Immediately after transfer (as practical) of the plasma aliquot, 10 μL of 50% acetic acid was added, the tube inverted to mix, and stored frozen at approximately −70° C. until analysis.

Urine Collection:

Urine samples were collected on wet ice at the intervals specified above into containers containing 0.6 g of citric acid. Urine samples were collected using steel pans placed under the cages. Upon completion of each collection interval, the urine was mixed, volume recorded, and transferred into four 20 mL aliquots and stored frozen at approximately −70° C. until analysis.

Analysis:

All plasma samples were analyzed using LC/MS/MS methods and pharmacokinetic analysis was performed using a noncompartmental method.

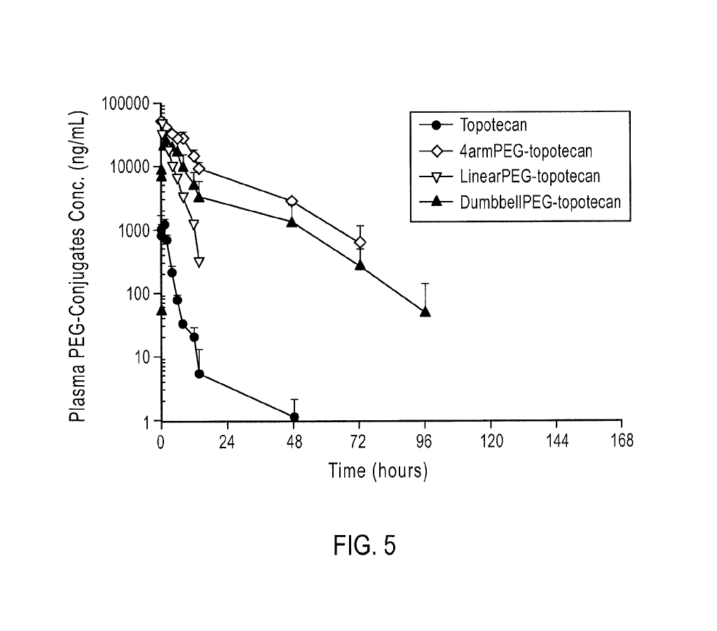

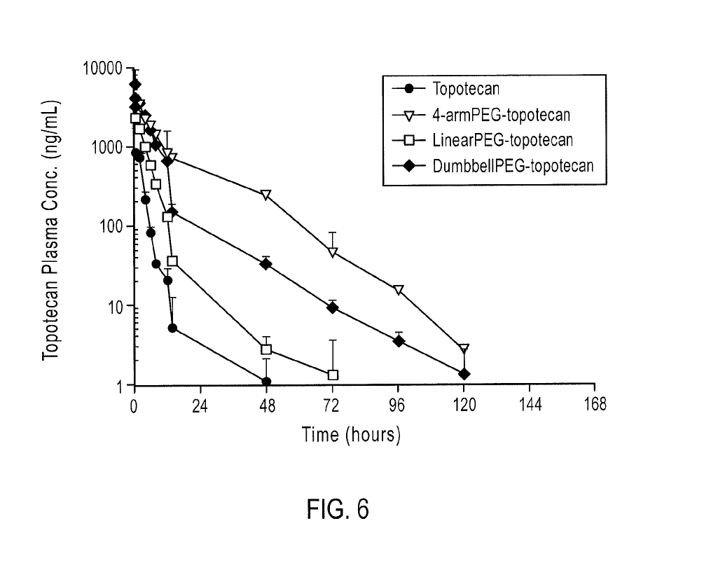

The results are plotted in FIG. 5 and in FIG. 6. As can be seen, the plots demonstrate the improved plasma half-lives of the conjugates in comparison to unconjugated topotecan, with both the 4-arm PEG topotecan and dumbbell PEG topotecan illustrating significantly longer circulating half-lives in plasma than topotecan per se, consistent with their advantageous anti-tumor activities as exemplified in the animal models investigated to date.

### Example 7

### Pharmacokinetic Analysis of Exemplary Topotecan Conjugates Following a Single IV Infusion in Beagle Dogs

It has been hypothesized that a PEGyation could facilitate enhanced drug concentrations within tumor tissues via an effect termed enhanced permeation and retention (EPR). To examine this effect for certain conjugates disclosed herein, a study was carried out in which concentrations of topotecan were measured in plasma and tumor-tissue from tumor bearing mice following a single administration of topotecan, dumbbell-PEG topotecan, or 4-arm-PEG topotecan.

Animal Care:

Six-weeks-old female athymic nude mice were purchased from Charles River (Wilmington, Mass.) and acclimated in the laboratories prior to experimentation. The animals were housed in microisolator cages, up to five per cage in a 12-hour light/dark cycle. The animals received filtered municipal water and sterilizable rodent diet (Harlan-Teklad TD8656) ad libitum. Cages were changed twice weekly. The animals were observed daily and clinical signs were noted.

Tumor Model:

Thirty-to-forty mg fragments of human NCI-H460 lung tumor were implanted sc in mice near the right axillary area using a 12-gauge trocar needle and allowed to grow. The day of tumor implantation was designated as day −7. Tumors were allowed to reach 100-234 mg in weight (100-234 mm³ in size) before the start of treatment. A sufficient number of mice were implanted so that tumors in a weight range as narrow as possible were selected for the trial on the day of treatment initiation (day 7 after tumor implantation). Those animals selected with tumors in the proper size range were assigned to the various treatment groups so that the median tumor weights on the first day of treatment were as close to each other as possible (137-154 mg).

Drug Formulation:

4-arm PEG topotecan was prepared at a concentration of 0.5 mg/mL solution of topotecan on each day of treatment by dissolving 15.15 mg of the compound per mL of saline. The lower dosages were achieved by further dilutions with saline. The dosing solutions were treated as light-sensitive (formulated under amber lights and dispensed into brown bottles), stored at room temperature, and were administered within 2 hours of formulation. Dumbbell PEG-topotecan was prepared at a concentration of 1.0 mglmL solution of topotecan on each day of treatment by dissolving 42.37 mg of the compound per mL of saline. The lower dosages were achieved by further dilutions with saline. The dosing solutions were treated as light-sensitive (formulated under amber lights and dispensed into brown bottles), stored at room temperature, and were administered within 2 hours of formulation. Topotecan was prepared at a concentration of 0.75 mglmL on each day of treatment in saline. The lower dosages were achieved by further dilutions with saline. The dosing solutions were treated as light-sensitive (formulated under amber lights and dispensed into brown bottles), were kept on ice, and were administered within 30 min of formulation. Saline was obtained from Phoenix Pharmaceutical, Inc.

Drug Treatment:

The experiment consisted of 3 treatment groups of 36 mice per group plus an untreated group of 3 mice for a total of 39 mice on the day of treatment. On the day of treatment (designated as day 1), topotecan (20 mg/kg), dumbbell-PEG-topotecan (30 mg/kg), or 4-arm-PEG-topotecan (20 mg/kg) were administered intravenously (iv). The above ‘topotecan-equivalent’ concentrations were calculated based on the topotecan content for each compound. The concentrations were MTD for each compound as determined in a single-dose MTD study, similar to the multi-dose MTD study described in Example 3, above. At intervals (0.5, 1, 2, 4, 8, 12, 24, 72, 120, 168, 216 hr) following drug administration, 3 animals per treatment group were sacrificed, blood collected via cardiac puncture, and tumor tissue was excised. Tumor tissue was flash frozen and stored at −80° C. until analysis. Blood was processed to obtain plasma and stabilizers added as described for PK studies in beagle dogs. Samples were frozen and stored at −80° C. until analysis. The untreated group of animals was included to provide tumor-growth measurements in the absence of treatment.

Tumor Measurements and Body Weights:

The sc tumors were measured and the animals were weighed twice weekly starting with the first day of treatment. Tumor volume was determined by caliper measurements (mm) and using the formula for an ellipsoid sphere: L×W²/2=mm³, where L and W refer to the larger and smaller perpendicular dimensions collected at each measurement. This formula is also used to calculate tumor weight, assuming unit density (1 mm³=1 mg).

Parameters Evaluated:

Plasma and tumor-tissue were analyzed by LC/MS-MS for topotecan. Data was reported as ng/mL for plasma samples and ng/mg for tumor samples.

Results:

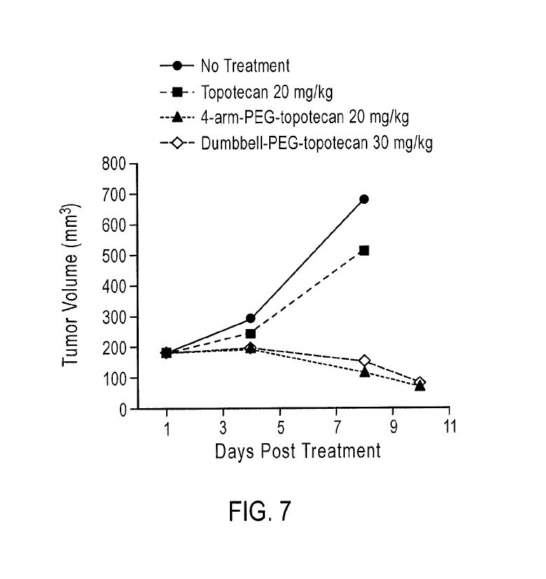

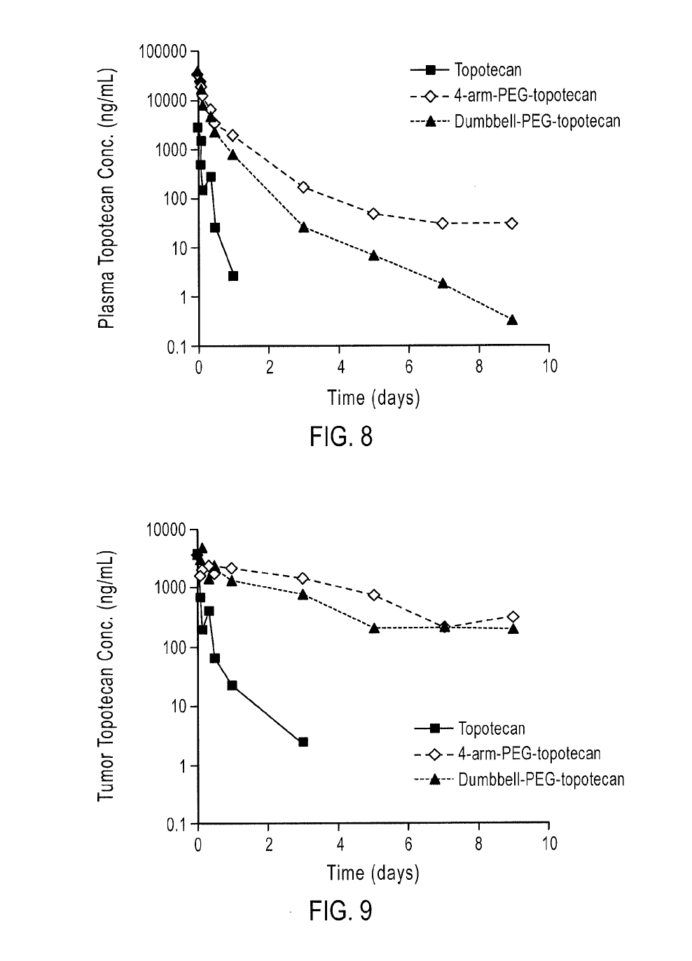

Tumors exhibited greater growth inhibition in animals treated with dumbbell-PEG-topotecan or 4-arm-PEG-topotecan than in animals treated with topotecan, which produced only minimal tumor growth delay using this administration schedule (FIG. 7). Measurable plasma concentrations of topotecan were detectable in both the dumbbell-PEG-topotecan and 4-arm-PEG-topotecan treated mice until the last time point sampled (day 9), while topotecan was undetectable beyond 24 hr post-injection in plasma of topotecan-treated mice (FIG. 8). Similarly, topotecan concentration in tumors from topotecan-treated mice declined to undetectable levels (<1 ng/mg) by day 5 post-injection (FIG. 9). Tumors from mice treated with dumbbell-PEG-topotecan or 4-arm-PEG-topotecan exhibited sustained topotecan concentrations (>100 ng/mg) through day 9 post injection (FIG. 9).

Summary:

Tumor growth was delayed modestly following a single iv injection of topotecan, but to a much greater degree by either dumbbell-PEG-topotecan or 4-arm-PEG-topotecan. In this experiment, a trend toward tumor regression was observed through day 10 following treatment with dumbbell-PEG-topotecan or 4-arm-PEG-topotecan. The data indicates that greater topotecan concentrations are maintained for many days in tumors following administration of dumbbell-PEG-topotecan or 4-arm-PEG-topotecan than following administration of topotecan. The improved suppression of tumor growth is consistent with increased duration of therapeutic concentrations of topotecan in the tumors. Such increased duration of topotecan within the tumors is consistent with an EPR effect for PEGylated-topotecans.

1. A water-soluble polymer conjugate of topotecan having two or more topotecan molecules covalently attached to a water-soluble polymer.

2. The water-soluble polymer conjugate of topotecan of claim 1, wherein the two or more topotecan molecules are releasably attached to a water-soluble polymer.

3. A water-soluble polymer conjugate of topotecan having a structure encompassed by the formula: 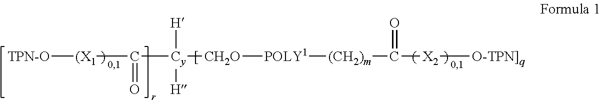
wherein:
y=0 or 1, such that when y=0, —C_{y}H′H″ is absent, and when y=1, C_{y} is present;
m is a positive integer from 1 to about 12;
X₁ and X₂, when present, are each an amino acid linker, such that the amino acid carboxyl carbon of the linker is adjacent to the TPN-oxygen (O);
each POLY¹ is a water-soluble, non-peptidic polymer;
q=1, 2, 3, or 4;
r=0 or 1; and
“TPN-O˜” corresponds to 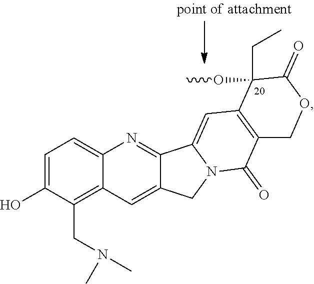
and pharmaceutically acceptable salts thereof,
where the following apply:
when r=1, q does not equal 4;
when r=0, q is selected from 2, 3, and 4, and
when r+q does not equal 4, then H′ and optionally H″ are present to bring the valence on C_{y} to four.

4. The water-soluble polymer conjugate of topotecan of claim 3, wherein both X₁ and X₂ are present.

5. The water-soluble polymer conjugate of topotecan of claim 3, wherein when X₁ or X₂ is absent.

6. The water-soluble polymer conjugate of topotecan of claim 3, wherein both X₁ and X₂ are absent.

7. The water-soluble polymer conjugate of topotecan of claim 3, wherein X₁ and X₂ are the same.

8. The water-soluble polymer conjugate of topotecan claim 3, wherein each amino acid linker comprises the structure —C(O)—CH(R″)—NH— wherein R″ is H, C1-C6 alkyl, or substituted C1-C6 alkyl.

9. The water-soluble polymer conjugate of topotecan of claim 3, wherein the amino acid linker corresponds to alanine, glycine, isoleucine, leucine, phenylalanine, and valine.

10. The water-soluble polymer conjugate of topotecan of claim 3, wherein r=1, q=1, and y=0, having the structure

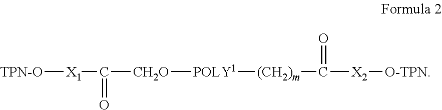

11. The water-soluble polymer conjugate of topotecan of claim 10, wherein m=1.

12. The water-soluble polymer conjugate of topotecan of claim 10, wherein X₁ and X₂ are both present.

13. The water-soluble polymer conjugate of topotecan of claim 10, wherein X₁ and X₂ are both glycine-based linkers, where X₁ corresponds to the formula: —C(O)CH₂NH— and X₂ corresponds to the formula: —NHCH₂C(O)—.

14. The water-soluble polymer conjugate of topotecan of claim 1, wherein the water soluble polymer conjugate corresponds to Formula 3: 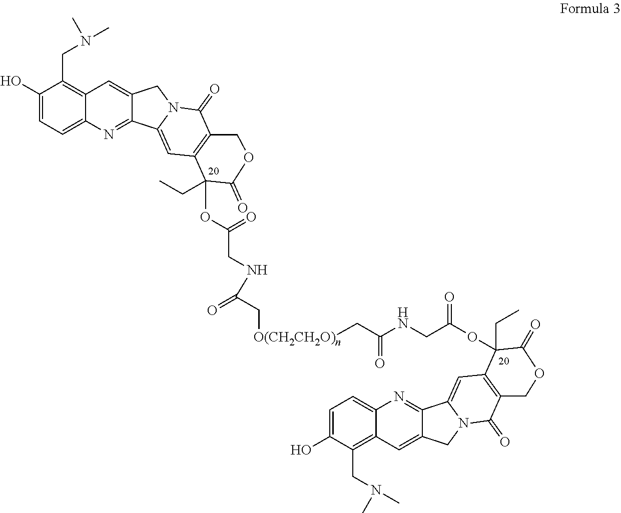
and pharmaceutically acceptable salts thereof, wherein the value of n ranges from about 10 to 1500.

15. The water-soluble polymer conjugate of topotecan of claim 14, wherein the value of n ranges from about 200 to about 800.

16. The water-soluble polymer conjugate of topotecan of claim 3, wherein r=0, y=1, q=4, and X₂ is present, such that the conjugate corresponds to Formula 4:

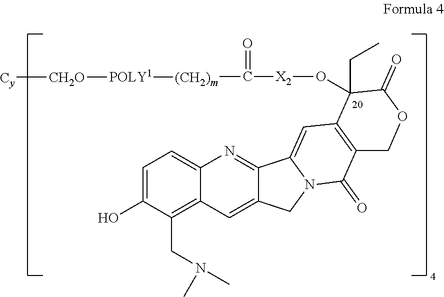

17. The water-soluble polymer conjugate of topotecan of claim 16, wherein m is equal to 1.

18. The water-soluble polymer conjugate of topotecan of claim 16, wherein X₂ corresponds to —NHCH₂C(O)—.

19. The water-soluble polymer conjugate of topotecan claim 3, wherein the conjugate corresponds to Formula 5: 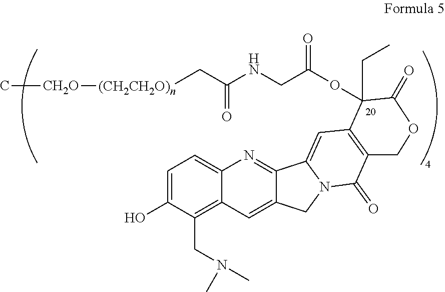
and pharmaceutically acceptable salts thereof, where n is a positive integer having a range from 10 to about 400.

20. The water-soluble polymer conjugate of topotecan of claim 3, wherein r=0, y=1, q=2, 3 or 4, and X₂ is absent, such that the conjugate corresponds to Formula 6: 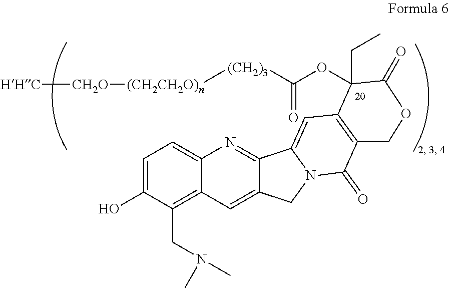
wherein n is a positive integer ranging from 10 to about 400, m is a positive integer from 3 to about 12, and when the number of polymer arms, —CH₂O—(CH₂CH₂O)ₙ—(CH₂)ₘ—C(O)—O-TPN covalently attached to central carbon CH′H″ is 2, then H′ and H″ is present to bring the valence on the central carbon to four; and when the number of polymer arms, —CH₂O—(CH₂CH₂O)ₙ—(CH₂)ₘ—C(O)—O-TPN covalently attached to central carbon CH′H″ is 3, then H′ is present to bring the valence on the central carbon to four, and pharmaceutically acceptable salts thereof.

21. A conjugate-containing composition comprising four-arm conjugates, wherein at least 80% of the four-arm conjugates in the composition have a structure encompassed by the formula: 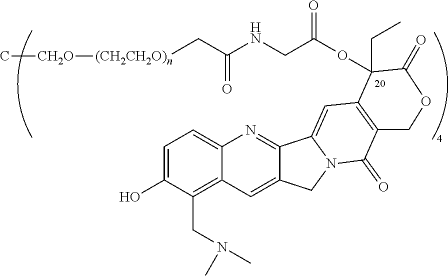
where n is a positive integer ranging from 10 to about 400, and pharmaceutically acceptable salts thereof.

22. The conjugate of claim 3, wherein POLY¹ represents a water-soluble and non-peptidic polymer selected from poly(alkylene glycol), poly(olefinic alcohol), poly(vinylpyrrolidone), poly(hydroxyalkylmethacrylamide), poly(hydroxyalkylmethacrylate), poly(saccharide), poly(α-hydroxy acid), poly(acrylic acid), poly(vinyl alcohol), polyphosphazene, polyoxazoline, poly(N-acryloylmorpholine), or copolymers or terpolymers thereof.

23. The conjugate of claim 22, wherein POLY¹ is a polyethylene glycol.

24. The conjugate of claim 22, wherein POLY¹ is a linear polyethylene glycol.

25. The water-soluble polymer conjugate of topotecan of claim 3, wherein r is equal to 1 and q is equal to 2, such that the conjugate corresponds to Formula 9: 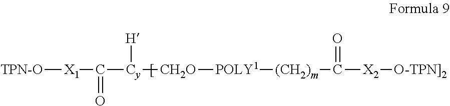
and pharmaceutically acceptable salts thereof.

26. The water-soluble polymer conjugate of topotecan of claim 24, wherein each POLY¹ is polyethylene glycol and X₁ and each X₂ correspond to the same amino acid.

27. The water-soluble polymer conjugate of topotecan of claim 25, wherein m is equal to 1.

28. The water-soluble polymer conjugate of topotecan of claim 3, wherein r is equal to 1 and q is equal to 3, such that the conjugate corresponds to Formula 10: 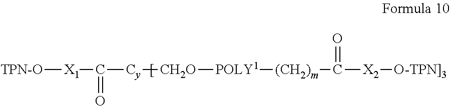
and pharmaceutically acceptable salts thereof.

29. The water-soluble polymer conjugate of topotecan of claim 28, wherein each POLY¹ is polyethylene glycol and X₁ and each X₂ correspond to the same amino acid.

30. The water-soluble polymer conjugate of topotecan of claim 28, wherein m is equal to 1.

31. The water-soluble polymer conjugate of topotecan of claim 3, wherein r is equal to 0 and q is equal to 2, such that the conjugate corresponds to Formula 11: 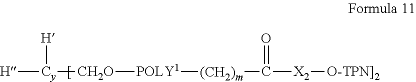
and pharmaceutically acceptable salts thereof.

32. The water-soluble polymer conjugate of topotecan of claim 31, wherein each POLY¹ is polyethylene glycol, and each X₂ corresponds to the same amino acid.

33. The water-soluble polymer conjugate of topotecan of claim 31, wherein each X₂ corresponds to glycine.

34. The water-soluble polymer conjugate of topotecan of claim 31, wherein m is equal to 1.

35. The water-soluble polymer conjugate of topotecan of claim 3, wherein r is equal to 0 and q is equal to 3, such that the conjugate corresponds to Formula 12: 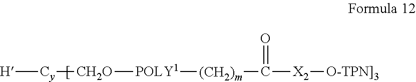
and pharmaceutically acceptable salts thereof.

36. The water-soluble polymer conjugate of topotecan of claim 35, wherein each POLY¹ is polyethylene glycol and each X₂ corresponds to the same amino acid.

37. The water-soluble polymer conjugate of topotecan of claim 35, wherein each X₂ corresponds to glycine.

38. The water-soluble polymer conjugate of topotecan of claim 35, wherein m is equal to 1.

39. A pharmaceutical composition comprising a topotecan conjugate of claim 1 and a pharmaceutically acceptable carrier.

40. A method comprising administering a topotecan conjugate of claim 1 to an individual.

41. A method of treating cancer or any other condition responsive to treatment with topotecan by administering a polymer conjugate of topotecan of claim 1.

42. A method comprising reacting a water-soluble, non-peptidic polymer structure having 2, 3 or 4 polymer arms, each individual polymer arm having a reactive carboxylic acid group or activated ester thereof at its terminus, with “q” moles or greater of a compound having the following structure, where “q” corresponds to the number of reactive carboxylic acid or activated ester functionalities in the reactive polymeric structure: 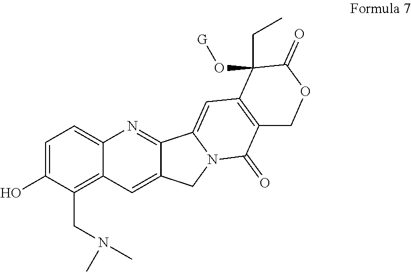
wherein G is H or —C(O)—CHR″—NH₂, where R″ is selected from H, C1-C6 alkyl, C1-C6 alkylaryl, and substituted C1-C6 alkyl.

43. The method of claim 42, wherein the polymer arms include a linear dumbbell like structure having a reactive carboxylic acid group or activated ester thereof at each polymer terminus.

44. The method of claim 42, wherein the C1-C6 alkyl groups are selected from —CH₃, —CH₂CHCH₃CH₃, —CH(CH₃)₂, —CH(CH₃)CH₂CH₃, and the substituted C1-C6 alkyl groups are selected from —(CH₂)₃—NH—C═N—NH₂, —CH₂CH₂COOH, —(CH₂)₄NH₂, —(CH₂)₂SCH₃, —CH₂-Ph, -p-CH₂C₆H₄—OH, —CH₂OH, and —CHCH₃OH, where the amino group thereof is optionally in protected form.
